(19) United States
(12) Patent Application Publication
(10) Pub. No.: US 2009/0238875 A1
(43) Pub. Date: Sep. 24, 2009

(54) Chitosan or Hyaluronic Acid-Poly(Ethylene Oxide)-and Chitosan-Hyaluronic Acid-Poly(Ethylene Oxide)-Based Hydrogel and Manufacturing Method Therefor

(75) Inventors: Insup Noh, Seoul (KR); Yongdoo Park, Seoul (KR); Kyuback Lee, Seoul (KR); Soonjung Hwang, Seoul (KR); Kyung Sun, Seoul (KR); Gunwoo Kim, Seoul (KR)

Correspondence Address: HARNESS, DICKEY & PIERCE, P.L.C., P.O. BOX 828, BLOOMFIELD HILLS, MI, 48303, US

(73) Assignee: Seoul National University of Technology Center for Industry Collaboration, Seoul (KR)

(21) Appl. No.: 12/083,705

(22) PCT Filed: Aug. 28, 2006
(86) PCT No.: PCT/KR2006/003383

§ 371 Date: Apr. 17, 2008

(30) Foreign Application Priority Data
Jan. 19, 2006 (KR) 10-2006-0005972

(51) Int. Cl. A61K 9/14; C07H 5/06

(52) U.S. Cl. 424/487; 536/55.1

(57) ABSTRACT

Disclosed are a chitosan-chitosan-polyethylene oxide hydrogel formed via covalent bonding between chitosan derivatives crosslinked with an acrylate or methacrylate functional group-containing substance and a thiol functional group-containing substance and hydrogel microbeads thereof; a hyaluronic acid-hyaluronic acid-polyethylene oxide hydrogel formed via covalent bonding between hyaluronic acid derivatives crosslinked with an acrylate or methacrylate functional group-containing substance and a thiol functional group-containing substance and hydrogel microbeads thereof; and a chitosan-hyaluronic acid-polyethylene oxide hydrogel formed via covalent bonding between a chitosan derivative crosslinked with a (meth)acrylate functional group-containing substance as well as a hyaluronic acid derivative crosslinked with a (meth) acrylate functional group-containing substance and a thiol functional group-containing substance and hydrogel microbeads thereof.

### TECHNICAL FIELD

The present invention relates to a chitosan- or hyaluronic acid-polyethylene oxide hydrogel and chitosan-hyaluronic acid-polyethylene oxide hydrogel, a bioactive substance delivery carrier and a scaffold for tissue engineering using the same, and methods for preparing the same. More particularly, the present invention relates to a chitosan-chitosan-polyethylene oxide hydrogel formed via covalent bonding between a chitosan derivative crosslinked with a substance having an acrylate or methacrylate functional group and a substance having a thiol functional group, a hyaluronic acid-hyaluronic acid-polyethylene oxide hydrogel formed via covalent bonding between a hyaluronic acid derivative crosslinked with a substance having an acrylate or methacrylate functional group and a substance having a thiol functional group, and a chitosan-hyaluronic acid-polyethylene oxide hydrogel formed via covalent bonding between a hyaluronic acid derivative crosslinked with a (meth)acrylate functional group as well as a chitosan derivative crosslinked with a (meth)acrylate functional group and a substance having a thiol functional group. Also, the present invention relates to a bioactive substance delivery carrier containing a bioactive substance supported thereon, and a scaffold for tissue engineering that allow cell adhesion to the chitosan-chitosan-polyethylene oxide hydrogel, the hyaluronic acid-hyaluronic acid-polyethylene oxide hydrogel and the chitosan-hyaluronic acid-polyethylene oxide hydrogel and degradation of the hydrogels. Further, the present invention relates to methods for preparing the chitosan-chitosan-polyethylene oxide hydrogel, the hyaluronic acid-hyaluronic acid-polyethylene oxide hydrogel and the chitosan-hyaluronic acid-polyethylene oxide hydrogel, as well as the bioactive substance delivery carrier.

### BACKGROUND ART

In general, chitosan is a natural polymeric substance having amino groups in its molecule, and is a deacetylated product of chitin obtained by treating chitin from crustacean shells in a high temperature and strong alkaline condition. Hyaluronic acid is prepared in the human body or microorganisms and is a natural polymer having free carboxylic acid groups in its molecule. Both chitosan and hyaluronic acid have been used widely in various chemical, medical and food engineering applications. Studies of chitosan have been focused on the production of scaffolds for tissue engineering. For example, studies that have been reported include “Biodegradable Polymeric Formulation for Tissue Engineering Surface-Coated with Chitosan and Preparation Thereof,” “Ionic Composite Scaffold Comprising Chitosan-Hyaluronic Acid,” “Bioabsorptive Nerve Guidance Channel and Preparation Thereof,” “Oligopeptides attached specifically to chondrocytes, Biodegradable Polymeric Substrate Comprising Extracellular Matrix for Manufacturing Artificial Organs and Preparation Thereof,” or the like. Additionally, studies of hyaluronic acid have been reported, such studies including “Temperature-Sensitive Degradable Hyaluronic Acid/Fluoronic Acid Composite Hydrogel for Controlled Release Delivery of Growth Factors,” “Crosslinked Hyaluronic Acid Hydrogel,”, “Hyaluronic acid/Type 2 Collagen Hydrogel,” “Preparation of Chitosan-Hyaluronic Acid Hybrid Scaffolds for Cartilage Regeneration,” or the like.

Although there have been intensive studies of hydrogels for use in various industrial applications, including medical, pharmaceutical, environmental engineering and cosmetic applications, for example, as drug or cell delivery carriers, or as scaffolds for tissue engineering including artificial skin, artificial cartilage, artificial bone, etc., improvements in mechanical properties of hydrogels, in a time required for preparing hydrogels, and in yield, activity and efficiency of a bioactive substance fixed to hydrogels are still required.

### DISCLOSURE OF THE INVENTION

Under these circumstances, the inventors of the present invention have prepared hyaluronic acid-acrylate, a chitosan-chitosan-polyethylene oxide hydrogel crosslinked with an acrylate- or methacrylate-containing substance, a hyaluronic acid-hyaluronic acid-polyethylene oxide hydrogel, a chitosan-hyaluronic acid-polyethylene oxide hydrogel crosslinked with an acrylate- or methacrylate-containing substance, and a microbead type hydrogel. In addition, the inventors of the present invention have found that the chitosan-chitosan-polyethylene oxide hydrogel, the hyaluronic acid-hyaluronic acid-polyethylene oxide hydrogel and the chitosan-hyaluronic acid-polyethylene oxide hydrogel can be used to effectively support bioactive substances such as peptides, proteins or cells thereon or to induce an effective chemical bonding of such bioactive substances, thereby improving yield and activity maintenance of the bioactive substances. The present invention is based on these findings.

It is an object of the present invention to provide a chitosan-chitosan-polyethylene oxide hydrogel formed via covalent bonding between a chitosan-chitosan derivative crosslinked with an acrylate- or methacrylate-containing substance and a thiol functional group-containing substance.

It is another object of the present invention to provide a hyaluronic acid-hyaluronic acid-polyethylene oxide hydrogel formed via covalent bonding between a hyaluronic acid-hyaluronic acid derivative crosslinked with an acrylate- or methacrylate-containing substance and a thiol functional group-containing substance.

It is still another object of the present invention to provide a chitosan-hyaluronic acid-polyethylene oxide hydrogel formed via covalent bonding between a chitosan-hyaluronic acid derivative crosslinked with an acrylate- or methacrylate-containing substance and a thiol functional group-containing substance.

It is still another object of the present invention to provide chitosan-chitosan-polyethylene oxide hydrogel, hyaluronic acid-hyaluronic acid-polyethylene oxide hydrogel and chitosan-hyaluronic acid-polyethylene oxide hydrogel in the form of microbeads.

It is still another object of the present invention to provide a scaffold for tissue engineering, which allow cell adhesion or anti-adhesion to the chitosan-chitosan-polyethylene oxide hydrogel, the hyaluronic acid-hyaluronic acid-polyethylene oxide hydrogel and the chitosan-hyaluronic acid-polyethylene oxide hydrogel and degradation of the hydrogels, so as to facilitate formation of a condition favorable to tissue regeneration, and a bioactive substance delivery carrier containing a bioactive substance supported thereon.

It is still another object of the present invention to provide a method for preparing a chitosan-chitosan-polyethylene oxide hydrogel, the method comprising the steps of: (a) providing an aqueous chitosan solution; (b) crosslinking chitosan with an acrylate functional group-containing substance to provide a chitosan derivative; (c) crosslinking chitosan with a methacrylate functional group-containing substance to provide a chitosan derivative; and (d) forming covalent bonds between a mixture of the chitosan derivatives and a thiol functional group-containing substance.

It is still another object of the present invention to provide a method for preparing a hyaluronic acid-hyaluronic acid-polyethylene oxide hydrogel, the method comprising the steps of: (a) providing an aqueous hyaluronic acid solution; (b) crosslinking hyaluronic acid with an acrylate functional group-containing substance to provide a hyaluronic acid derivative; (c) crosslinking hyaluronic acid with a methacrylate functional group-containing substance to provide a hyaluronic acid derivative; and (d) forming covalent bonds between a mixture of the hyaluronic acid derivatives and a thiol functional group-containing substance.

It is still another object of the present invention to provide a method for preparing a chitosan-hyaluronic acid-polyethylene oxide hydrogel, the method comprising the steps of: (a) providing an aqueous chitosan solution and an aqueous hyaluronic acid solution; (b) crosslinking chitosan with an acrylate or methacrylate functional group-containing substance to provide a chitosan derivative; (c) crosslinking hyaluronic acid with an acrylate or methacrylate functional group-containing substance to provide a hyaluronic acid derivative; and (d) forming covalent bonds between a mixture of the chitosan derivative with the hyaluronic acid derivative and a thiol functional group-containing substance.

It is still another object of the present invention to provide a method for preparing chitosan-chitosan-polyethylene oxide hydrogel microbeads, the method comprising the steps of: (a) providing an aqueous chitosan solution; (b) crosslinking chitosan with an acrylate functional group-containing substance to provide a chitosan derivative; (c) crosslinking chitosan with a methacrylate functional group-containing substance to provide a chitosan derivative; (d) forming a mixed solution containing a mixture of the chitosan derivatives and a thiol functional group-containing substance; (e) adding the mixed solution dropwise to a solution containing a hydrophobic solvent and a surfactant and dispersing the mixed solution therein; and (f) allowing the chitosan derivatives and polyethylene oxide dispersed in the solution to form hydrogel microbeads and recovering the microbeads.

It is still another object of the present invention to provide a method for preparing hyaluronic acid-hyaluronic acid-polyethylene oxide hydrogel microbeads, the method comprising the steps of: (a) providing an aqueous hyaluronic acid solution; (b) crosslinking hyaluronic acid with an acrylate functional group-containing substance to provide a hyaluronic acid derivative; (c) crosslinking hyaluronic acid with a methacrylate functional group-containing substance to provide a hyaluronic acid derivative; (d) forming a mixed solution containing a mixture of the hyaluronic acid derivatives and a thiol functional group-containing substance; (e) adding the mixed solution dropwise to a solution containing a hydrophobic solvent and a surfactant and dispersing the mixed solution therein; and (f) allowing the hyaluronic acid derivatives and polyethylene oxide dispersed in the solution to form hydrogel microbeads and recovering the microbeads.

It is still another object of the present invention to provide a method for preparing chitosan-hyaluronic acid-polyethylene oxide hydrogel microbeads, the method comprising the steps of: (a) providing an aqueous chitosan solution and an aqueous hyaluronic acid solution; (b) crosslinking chitosan with an acrylate or methacrylate functional group-containing substance to provide a chitosan derivative; (c) crosslinking hyaluronic acid with an acrylate or methacrylate functional group-containing substance to provide a hyaluronic acid derivative; (d) forming a mixed solution containing a mixture of the chitosan derivative with the hyaluronic acid derivative and a thiol functional group-containing substance; (e) adding the mixed solution dropwise to a solution containing a hydrophobic solvent and a surfactant and dispersing the mixed solution therein; and (f) allowing the chitosan derivative, the hyaluronic acid derivative and polyethylene oxide dispersed in the solution to form hydrogel microbeads and recovering the microbeads.

It is still another object of the present invention to provide a method for preparing a bioactive substance delivery carrier or a scaffold for tissue engineering, the method comprising the steps of: (a) providing an aqueous chitosan solution; (b) crosslinking chitosan with an acrylate functional group-containing substance to provide a chitosan derivative; (c) crosslinking chitosan with a methacrylate functional group-containing substance to provide a chitosan derivative; (d) mixing a bioactive substance with the chitosan derivatives or a thiol functional group-containing substance; and (e) forming covalent bonds between the chitosan derivatives and the thiol functional group-containing substance while the bioactive substance is supported thereon.

It is still another object of the present invention to provide a method for preparing a bioactive substance delivery carrier or a scaffold for tissue engineering, the method comprising the steps of: (a) providing an aqueous hyaluronic acid solution; (b) crosslinking hyaluronic acid with an acrylate functional group-containing substance to provide a hyaluronic acid derivative; (c) crosslinking hyaluronic acid with a methacrylate functional group-containing substance to provide a hyaluronic acid derivative; (d) mixing a bioactive substance with the hyaluronic acid derivatives or a thiol functional group-containing substance; and (e) forming covalent bonds between the hyaluronic acid derivatives and the thiol functional group-containing substance while the bioactive substance is supported thereon.

It is still another object of the present invention to provide a method for preparing a bioactive substance delivery carrier or a scaffold for tissue engineering, the method comprising the steps of: (a) providing an aqueous chitosan solution and an aqueous hyaluronic acid solution; (b) crosslinking chitosan with an acrylate or methacrylate functional group-containing substance to provide a chitosan derivative; (c) crosslinking hyaluronic acid with an acrylate or methacrylate functional group-containing substance to provide a hyaluronic acid derivative; (d) mixing a bioactive substance with the chitosan derivative and the hyaluronic acid derivative, or with a thiol functional group-containing substance; and (e) forming covalent bonds between the chitosan derivative as well as the hyaluronic acid derivative and the thiol functional group-containing substance while the bioactive substance is supported thereon.

It is still another object of the present invention to provide a method for preparing a bioactive substance delivery carrier or a scaffold for tissue engineering, the method comprising the steps of: (a) providing an aqueous chitosan solution; (b) crosslinking chitosan with an acrylate functional group-containing substance to provide a chitosan derivative; (c) crosslinking chitosan with a methacrylate functional group-containing substance to provide a chitosan derivative; (d) mixing a bioactive substance with the chitosan derivatives or a thiol functional group-containing substance to provide a mixed solution; (e) adding the mixed solution dropwise to a solution containing a hydrophobic solvent and a surfactant and dispersing the mixed solution therein; and (f) allowing the chitosan and polyethylene oxide dispersed in the solution to form hydrogel microbeads and recovering the microbeads.

It is still another object of the present invention to provide a method for preparing a bioactive substance delivery carrier or a scaffold for tissue engineering, the method comprising the steps of: (a) providing an aqueous hyaluronic acid solution; (b) crosslinking hyaluronic acid with an acrylate functional group-containing substance to provide a hyaluronic acid derivative; (c) crosslinking hyaluronic acid with a methacrylate functional group-containing substance to provide a hyaluronic acid derivative; (d) mixing a bioactive substance with the hyaluronic acid derivatives or a thiol functional group-containing substance to provide a mixed solution; (e) adding the mixed solution dropwise to a solution containing a hydrophobic solvent and a surfactant and dispersing the mixed solution therein; and (f) allowing the hyaluronic acid derivatives and polyethylene oxide dispersed in the solution to form hydrogel microbeads and recovering the microbeads.

It is yet another object of the present invention to provide a bioactive substance delivery carrier or a scaffold for tissue engineering, the method comprising the steps of: (a) providing an aqueous chitosan solution and an aqueous hyaluronic acid solution; (b) crosslinking chitosan with an acrylate or methacrylate functional group-containing substance to provide a chitosan derivative; (c) crosslinking hyaluronic acid with an acrylate or methacrylate functional group-containing substance to provide a hyaluronic acid derivative; (d) mixing a bioactive substance with the chitosan derivative and the hyaluronic acid derivative, or with a thiol functional group-containing substance; (e) further mixing the chitosan derivative and the hyaluronic acid derivative with the thiol functional group-containing substance to provide a mixed solution while the bioactive substance is supported thereon; (f) adding the mixed solution dropwise to a solution containing a hydrophobic solvent and a surfactant and dispersing the mixed solution therein; and (g) allowing the chitosan derivative, the hyaluronic acid derivative and polyethylene oxide dispersed in the solution to form hydrogel microbeads and recovering the microbeads.

To accomplish the above-described objects, according to the present invention, the chitosan-chitosan-polyethylene oxide hydrogel, the hyaluronic acid-hyaluronic acid-polyethylene oxide hydrogel and the chitosan-hyaluronic acid-polyethylene oxide hydrogel, and the methods for preparing the same are characterized as follows:

According to the first aspect of the present invention, there is provided a chitosan-chitosan-polyethylene oxide hydrogel formed via covalent bonding between a chitosan derivative crosslinked with a methacrylate functional group-containing substance as well as a chitosan derivative crosslinked with an acrylate functional group-containing substance and a thiol functional group-containing substance.

According to the second aspect of the present invention, there is provided a hyaluronic acid-hyaluronic acid-polyethylene oxide hydrogel formed via covalent bonding between a hyaluronic acid derivative crosslinked with an acrylate functional group-containing substance as well as a hyaluronic acid derivative crosslinked with a methacrylate functional group-containing substance and a thiol functional group-containing substance.

According to the third aspect of the present invention, there is provided a chitosan-hyaluronic acid-polyethylene oxide hydrogel formed via covalent bonding between a chitosan derivative crosslinked with an acrylate or methacrylate functional group-containing substance as well as a hyaluronic acid derivative crosslinked with an acrylate or methacrylate functional group-containing substance and a thiol functional group-containing substance.

According to the fourth aspect of the present invention, there is provided the above chitosan-chitosan-polyethylene oxide hydrogel, hyaluronic acid-hyaluronic acid-polyethylene oxide hydrogel and chitosan-hyaluronic acid-polyethylene oxide hydrogel in the form of microbeads.

According to the fifth aspect of the present invention, there is provided a bioactive substance delivery carrier comprising a bioactive substance supported on the above chitosan-chitosan-polyethylene oxide hydrogel, hyaluronic acid-hyaluronic acid-polyethylene oxide hydrogel and chitosan-hyaluronic acid-polyethylene oxide hydrogel.

According to the sixth aspect of the present invention, there is provided a scaffold for tissue engineering, which comprises a hydrogel or microbeads to which a bioactive substance is chemically or physically bound, the hydrogel or microbeads being formed of chitosan-chitosan-polyethylene oxide, hyaluronic acid-hyaluronic acid-polyethylene oxide, chitosan-hyaluronic acid-polyethylene oxide and chitosan-hyaluronic acid-polyethylene oxide-peptide

According to the seventh aspect of the present invention, there is provided a method for preparing a chitosan acrylate-chitosan acrylate-polyethylene oxide hydrogel, chitosan methacrylate-chitosan methacrylate-polyethylene oxide hydrogel and chitosan acrylate-chitosan methacrylate-polyethylene oxide hydrogel, the method comprising the steps of: (a) providing an aqueous chitosan solution; (b) crosslinking chitosan with an acrylate functional group-containing substance to provide a chitosan-acrylate derivative; (c) crosslinking chitosan with a methacrylate functional group-containing substance to provide a chitosan-methacrylate derivative; and (d) forming covalent bonds between a mixture of the chitosan acrylate derivatives, a mixture of the chitosan methacrylate derivative or a mixture of the chitosan acrylate derivative with the chitosan methacrylate derivative and a thiol functional group-containing substance.

According to the eighth aspect of the present invention, there is provide a method for preparing chitosan acrylate-chitosan acrylate-polyethylene oxide microbeads, chitosan methacrylate-chitosan methacrylate-polyethylene oxide microbeads and chitosan acrylate-chitosan methacrylate-polyethylene oxide microbeads the method comprising the steps of: (a) providing an aqueous chitosan solution; (b) crosslinking chitosan with an acrylate functional group-containing substance to provide a chitosan-acrylate derivative; (c) crosslinking chitosan with a methacrylate functional group-containing substance to provide a chitosan-methacrylate derivative; (d) forming a mixed solution containing a mixture of the chitosan acrylate or chitosan methacrylate derivatives and a thiol functional group-containing substance; (e) adding the mixed solution dropwise to a solution containing a hydrophobic solvent and a surfactant and dispersing the mixed solution therein; and (f) allowing the chitosan derivatives and polyethylene oxide dispersed in the solution to form hydrogel microbeads and recovering the microbeads.

According to the ninth aspect of the present invention, there is provided a method for preparing chitosan-polyethylene oxide microbeads containing a bioactive substance, the method comprising the steps of: (a) providing an aqueous chitosan solution; (b) crosslinking chitosan with an acrylate functional group-containing substance to provide a chitosan derivative; (c) crosslinking chitosan with a methacrylate functional group-containing substance to provide a chitosan derivative; (d) incorporating a bioactive substance into a mixture of the chitosan derivatives or a thiol functional group-containing substance and mixing the chitosan derivatives and the thiol functional group-containing substance to provide a mixed solution; (e) adding the mixed solution containing the bioactive substance dropwise to a solution containing a hydrophobic solvent and a surfactant and dispersing the mixed solution therein; and (f) allowing the chitosan derivatives and polyethylene oxide dispersed in the solution to form hydrogel microbeads and recovering the microbeads.

According to the tenth aspect of the present invention, there is provided a method for preparing hyaluronic acid acrylate-polyethylene oxide hydrogel, the method comprising the steps of: (a) forming an aqueous hyaluronic acid solution; (b) crosslinking hyaluronic acid in the aqueous solution with an acrylate functional group-containing substance to form a hyaluronic acid-acrylate derivative; and (c) forming covalent bonds between the hyaluronic acid-acrylate derivative and a thiol functional group-containing substance.

According to eleventh aspect of the present invention, there is provide a method for preparing hyaluronic acid-acrylate, the method comprising the steps of: (a) forming an aqueous hyaluronic acid solution; (b) forming an adipic acid diamide solution; (c) chemically combining adipic acid dihydrazide with tert-butyl group-containing di-tert-butyldicarbonate; (d) separating adipic acid hydrazide butyl carbonate from the chemical bond forming step; (e) allowing the adipic acid hydrazide butyl carbonate to react with hyaluronic acid to provide hyaluronic acid-adipic acid hydrazide butyl carbonate; (f) performing a chemical reaction between the hyaluronic acid-adipic acid hydrazide butyl carbonate with hyaluronic acid to provide hyaluronic acid-adipic acid-butyl carbonate; (g) removing a terminal butyl group from the hyaluronic acid-adipic acid-butyl carbonate to form hyaluronic acid-adipic acid, followed by separation; (h) chemically combining the hyaluronic acid-adipic acid with acrylic acid to provide hyaluronic acid-adipic acid-acrylate (hyaluronic acid-acrylate); and (i) removing unreacted acrylic acid to separate hyaluronic acid-acrylate.

According to the twelfth aspect of the present invention, there is provide a method for preparing a hyaluronic acid-hyaluronic acid-polyethylene oxide hydrogel, the method comprising the steps of: (a) providing an aqueous hyaluronic acid solution; (b) crosslinking hyaluronic acid with an acrylate functional group-containing substance to provide a hyaluronic acid derivative; (c) crosslinking hyaluronic acid with a methacrylate functional group-containing substance to provide a hyaluronic acid derivative; and (d) forming covalent bonds between a mixture of the hyaluronic acid derivatives and a thiol functional group-containing substance.

According to the thirteenth aspect of the present invention, there is provided a method for preparing hyaluronic acid-hyaluronic acid-polyethylene oxide microbeads, the method comprising the steps of: (a) providing an aqueous hyaluronic acid solution; (b) crosslinking hyaluronic acid with an acrylate functional group-containing substance to provide a hyaluronic acid derivative; (c) crosslinking hyaluronic acid with a methacrylate functional group-containing substance to provide a hyaluronic acid derivative; (d) mixing a mixture of the hyaluronic acid derivatives with a thiol functional group-containing substance to provide a mixed solution; (e) adding the mixed solution dropwise to a solution containing a hydrophobic solvent and a surfactant and dispersing the mixed solution therein; and (f) allowing the hyaluronic acid derivatives and polyethylene oxide dispersed in the solution to form hydrogel microbeads and recovering the microbeads.

According to the fourteenth aspect of the present invention, there is provided a method for preparing hyaluronic acid acrylate-polyethylene oxide microbeads or hyaluronic acid methacrylate-polyethylene oxide microbeads containing a bioactive substance, the method comprising the steps of: (a) providing an aqueous hyaluronic acid solution; (b) crosslinking hyaluronic acid with an acrylate functional group-containing substance to provide a hyaluronic acid-acrylate derivative; (c) crosslinking hyaluronic acid with a methacrylate functional group-containing substance to provide a hyaluronic acid-methacrylate derivative; (d) incorporating a bioactive substance into a mixture of the hyaluronic acid-acrylate derivatives, a mixture of the hyaluronic acid-methacrylate derivatives, or into a thiol functional group-containing polyethylene oxide solution, and mixing the hyaluronic acid derivatives and the thiol functional group-containing substance to provide a mixed solution; (e) adding the mixed solution containing the bioactive substance dropwise to a solution containing a hydrophobic solvent and a surfactant and dispersing the mixed solution therein; and (f) allowing the hyaluronic acid derivatives and polyethylene oxide dispersed in the solution to form hydrogel microbeads and recovering the microbeads.

According to the fifteenth aspect of the present invention, there is provided a method for preparing a chitosan-hyaluronic acid-polyethylene oxide hydrogel, the method comprising the steps of: (a) providing an aqueous chitosan solution and an aqueous hyaluronic acid solution, individually; (b) crosslinking chitosan with an acrylate or methacrylate functional group-containing substance to provide a chitosan derivative; (c) crosslinking hyaluronic acid with an acrylate or methacrylate functional group-containing substance to provide a hyaluronic acid derivative; and (d) forming covalent bonds between a mixture of the chitosan derivative with the hyaluronic acid derivative and a thiol functional group-containing substance.

According to the sixteenth object of the present invention, there is provided a method for preparing chitosan-hyaluronic acid-polyethylene oxide microbeads, the method comprising the steps of: (a) providing an aqueous chitosan solution and an aqueous hyaluronic acid solution, individually; (b) crosslinking chitosan with an acrylate or methacrylate functional group-containing substance to provide a chitosan derivative; (c) crosslinking hyaluronic acid with an acrylate or methacrylate functional group-containing substance to provide a hyaluronic acid derivative; (d) forming a mixed solution containing a mixture of the chitosan derivative with the hyaluronic acid derivative and a thiol functional group-containing substance; (e) adding the mixed solution dropwise to a solution containing a hydrophobic solvent and a surfactant and dispersing the mixed solution therein; and (f) allowing the chitosan derivative, the hyaluronic acid derivative and polyethylene oxide dispersed in the solution to form hydrogel microbeads and recovering the microbeads.

According to the seventeenth aspect of the present invention, there is provided a method for preparing a bioactive substance delivery carrier, the method comprising the steps of: (a) providing an aqueous chitosan solution and an aqueous hyaluronic acid solution, individually; (b) crosslinking chitosan with an acrylate or methacrylate functional group-containing substance to provide a chitosan derivative; (c) crosslinking hyaluronic acid with an acrylate or methacrylate functional group-containing substance to provide a hyaluronic acid derivative; (d) mixing a bioactive substance with the chitosan derivative and the hyaluronic acid derivative or with a thiol functional group-containing substance; and (e) forming covalent bonds between the chitosan derivative as well as the hyaluronic acid derivative and the thiol functional group-containing substance while the bioactive substance is supported thereon.

According to the eighteenth aspect of the present invention, there is provided a method for preparing chitosan acrylate-hyaluronic acid acrylate-polyethylene oxide microbeads, chitosan acrylate-hyaluronic acid methacrylate-polyethylene oxide microbeads, chitosan methacrylate-hyaluronic acid acrylate-polyethylene oxide microbeads, or chitosan methacrylate-hyaluronic acid methacrylate-polyethylene oxide microbeads containing a bioactive substance delivery carrier, the method comprising the steps of: (a) providing an aqueous chitosan solution and an aqueous hyaluronic acid solution; (b) crosslinking chitosan with an acrylate or methacrylate functional group-containing substance to provide a chitosan-acrylate derivative or a chitosan-methacrylate derivative; (c) crosslinking hyaluronic acid with an acrylate or methacrylate functional group-containing substance to provide a hyaluronic acid-acrylate derivative or a hyaluronic acid-methacrylate derivative; (d) incorporating a bioactive substance into the chitosan-acrylate derivative solution or the chitosan-methacrylate derivative solution, the hyaluronic acid-acrylate derivative solution or the hyaluronic acid-methacrylate derivative solution, or into a thiol functional group-containing polyethylene oxide solution, and mixing the chitosan-acrylate derivative or the chitosan-methacrylate derivative and the hyaluronic acid-acrylate derivative or the hyaluronic acid-methacrylate derivative with the thiol functional group-containing polyethylene oxide solution while the bioactive substance is supported thereon to provide a mixed solution; (e) adding the mixed solution containing the bioactive substance dropwise to a solution containing a hydrophobic solvent and a surfactant and dispersing the mixed solution therein; and (f) allowing the chitosan-acrylate derivative or the chitosan-methacrylate derivative, the hyaluronic acid-acrylate derivative or the hyaluronic acid-methacrylate derivative and polyethylene oxide dispersed in the solution to form hydrogel microbeads and recovering the microbeads.

### ADVANTAGEOUS EFFECTS

According to the present invention, the chitosan-chitosan-polyethylene oxide hydrogel, the hyaluronic acid-hyaluronic acid-polyethylene oxide hydrogel and the chitosan-hyaluronic acid-polyethylene oxide hydrogel can be used to effectively support physiologically substances such as peptides, proteins or cells thereon or to induce an effective chemical bonding of such bioactive substances, thereby improving yield and activity maintenance of the bioactive substances.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a reaction scheme representing a preferred embodiment of the preparation of the chitosan-methacrylate compound according to the present invention;

FIG. 2 is a reaction scheme representing a preferred embodiment of the preparation of the chitosan-acrylate compound according to the present invention;

FIG. 3 is a reaction scheme representing a preferred embodiment of the preparation of the hyaluronic acid-methacrylate compound according to the present invention;

FIG. 4 is a reaction scheme representing a preferred embodiment of the preparation of hyaluronic acid-adipic dihydrazide (HA-ADH-BOC) containing hyaluronic acid protected with tert-butyl group;

FIG. 5 is a reaction scheme representing a preferred embodiment of the preparation of the hyaluronic acid-adipic acid-acrylate compound (hyaluronic acid-acrylate: HA-Ac) compound via the reaction between hyaluronic acid-adipic acid hydrazide (HA-ADH), from which tert-butyl group is removed, and acrylic acid;

FIG. 6 is a reaction scheme (A) representing a preferred embodiment of the preparation of the chitosan (or hyaluronic acid)-polyethylene oxide hydrogel according to the present invention, and a schematic view (B) showing a network structure of the chitosan-hyaluronic acid-polyethylene oxide hydrogel according to a preferred embodiment of the present invention;

FIG. 7 is the NMP spectrum of a chitosan derivative according to a preferred embodiment of the present invention, wherein (A) represents chitosan-acrylate, (B) represents chitosan-methacrylate, and (C) represents chitosan;

FIG. 8 is the NMR spectrum of a hyaluronic acid derivative according to a preferred embodiment of the present invention, wherein (A) represents hyaluronic acid, (B) represents hyaluronic acid-adipic acid hydrazide tert-butyl hydrazide compound protected with tert-butyl group, and (C) represents a hyaluronic acid-adipic acid-acrylate compound (hyaluronic acid-acrylate: HA-Ac);

FIG. 9 is the rheology graph of a chitosan-hyaluronic acid-polyethylene oxide hydrogel according to a preferred embodiment of the present invention, wherein (A) represents a hydrogel using 100% of chitosan-acrylate, (B) represents a hydrogel using 75% of chitosan-acrylate and 25% of hyaluronic acid-aminopropyl methacrylate, (C) represents a hydrogel using 50% of chitosan-acrylate and 50% of hyaluronic acid-aminopropyl methacrylate, and (D) represents the rheology graph of a hyaluronic acid-polyethylene oxide hydrogel using hyaluronic acid-adipic acid-acrylate;

FIG. 10 is a graph showing the results of cell growth obtained by observing smooth muscle cells 6 hours and 3 days after culturing the cells on a chitosan-polyethylene oxide hydrogel;

FIG. 11 is a photographic view taken by an optical microscope, which shows the results obtained by carrying out cell culture for 6 hours on a hydrogel prepared by using 100% chitosan, a hyaluronic acid-chitosan hydrogel prepared by using 75% of hyaluronic acid (HA) and 25% of chitosan, a chitosan-hyaluronic acid hydrogel prepared by using 50% of hyaluronic acid (HA) and 50% of chitosan, and on a hydrogel prepared by using 25% of hyaluronic acid (HA) and 75% of chitosan, according to the present invention;

FIG. 12 is a photographic view taken by an optical microscope, which shows the results obtained by carrying out cell culture for 3 days on a chitosan hydrogel prepared by using 100% chitosan, a chitosan-hyaluronic acid hydrogel prepared by using 25% of hyaluronic acid (HA) and 75% of chitosan, a chitosan-hyaluronic acid hydrogel prepared by using 50% of hyaluronic acid (HA) and 50% of chitosan, and on a polystyrene cell culture flask; and

FIG. 13 is a photographic view showing hyaluronic acid-polyethylene oxide microbeads obtained by using a mixed solution of hyaluronic acid-hyaluronic acid-polyethylene oxide prepared by mixing 50% of hyaluronic acid-acrylate solution with 50% of hyaluronic acid-methacrylate solution, and further mixing the resultant mixture with a polyethylene oxide solution, wherein (A) is taken by an optical microscope and (B) is taken by an electron microscope.

### BEST MODE FOR CARRYING OUT THE INVENTION

Hereinafter, preferred embodiments of the present invention will be described. For the purposes of clarity and simplicity, a detailed description of known functions and configurations incorporated herein will be omitted as it may make the subject matter of the present invention unclear.

The present invention provides a chitosan-chitosan-polyethylene oxide hydrogel and hydrogel microbeads formed via covalent bonding between a chitosan derivative crosslinked with an acrylate-containing substance as well a chitosan derivative crosslinked with a methacrylate-containing substance and a thiol functional group-containing substance; a hyaluronic acid-hyaluronic acid-polyethylene oxide hydrogel and hydrogel microbeads formed via covalent bonding between a hyaluronic acid derivative crosslinked with an acrylate-containing substance as well a hyaluronic acid derivative crosslinked with a methacrylate-containing substance and a thiol functional group-containing substance; and a chitosan-hyaluronic acid-polyethylene oxide hydrogel and microbeads formed via covalent bonding between a hyaluronic acid derivative crosslinked with an acrylate- or methacrylate-containing substance as well as a chitosan derivative crosslinked with an acrylate- or methacrylate-containing substance and a thiol functional group-containing substance.

As used herein, the term “hydrogel” means a three-dimensional structure of a polymer containing a sufficient amount of water. In view of the objects of the present invention, the hydrogel includes a chitosan-chitosan-polyethylene oxide hydrogel, a hyaluronic acid-hyaluronic acid-polyethylene oxide hydrogel and a chitosan-hyaluronic acid-polyethylene oxide hydrogel. First, an aqueous chitosan or hyaluronic acid is chemically combined with an acrylate or methacrylate group-containing molecule to form chitosan-acrylate or chitosan-methacrylate, or hyaluronic acid-acrylate or hyaluronic acid-methacrylate. Next, the acryl or methacryl groups of a mixture of the chitosan-acrylate and the chitosan-methacrylate are allowed to be bonded with a thiol functional group-containing polyethylene oxide, the acryl or methacryl groups of a mixture of the hyaluronic acid-acrylate and the hyaluronic acid-methacrylate are allowed to be bonded with a thiol functional group-containing polyethylene oxide, and the acryl or methacryl groups of a mixture of the chitosan-acrylate and the hyaluronic acid-methacrylate are allowed to be bonded with a thiol functional group-containing polyethylene oxide to provide a hydrogel and hydrogel microbeads.

As used herein, the term “hydrogel beads” means a hydrogel having the above-mentioned hydrogel characteristics and provided in the form of micro-sized beads. According to the particular process for preparing the beads, the hydrogel beads may be controlled to have a micro size or a sub-micro size.

Chitosan used in the present invention is deacetylated chitosan, preferably aqueous chitosan deacetylated to 60% or more, and more preferably aqueous chitosan deacetylated to about 85%. Additionally, chitosan has a size of 1-1,000 KDa, preferably 5 KDa˜200 KDa. Chitosan has excellent bio-affinity and low antigenic activity and is degraded in vivo to be discharged from the human body, and thus is preferred as a medical material.

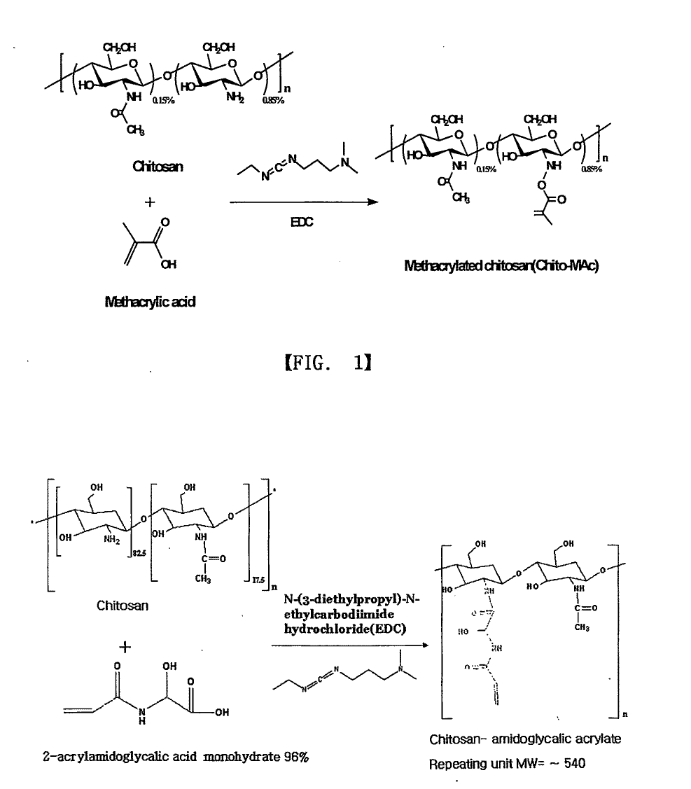

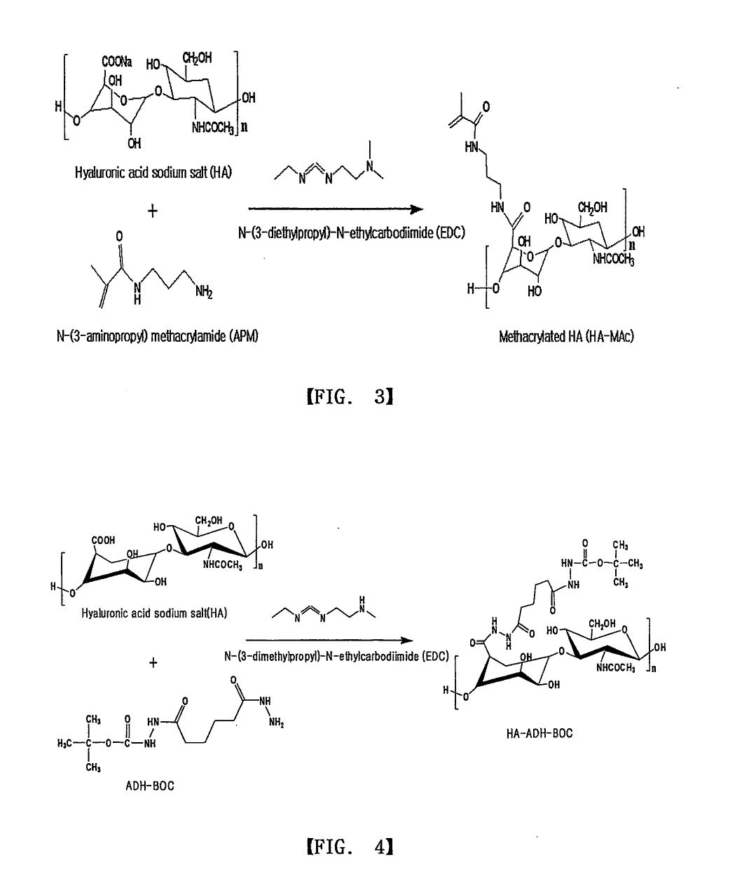

Chitosan used for preparing the hydrogel and microbeads according to the present invention is an acrylate- or methacrylate-containing chitosan derivative, formed via crosslinking between the amine functional groups of chitosan and carboxyl functional groups of acrylate or methacrylate. According to a preferred embodiment of the present invention, chitosan-methacrylate and chitosan-acrylate compounds are obtained via the reaction schemes as shown in FIGS. 1 and 2.

Preferably, hyaluronic acid used in the present invention is aqueous hyaluronic acid. Hyaluronic acid has a size of 1˜3,000 KDa, more preferably 5 KDa˜500 KDa. Hyaluronic acid has excellent bio-affinity and low antigenic property and is degraded in vivo to be discharged from the human body, and thus is preferred as a medical material.

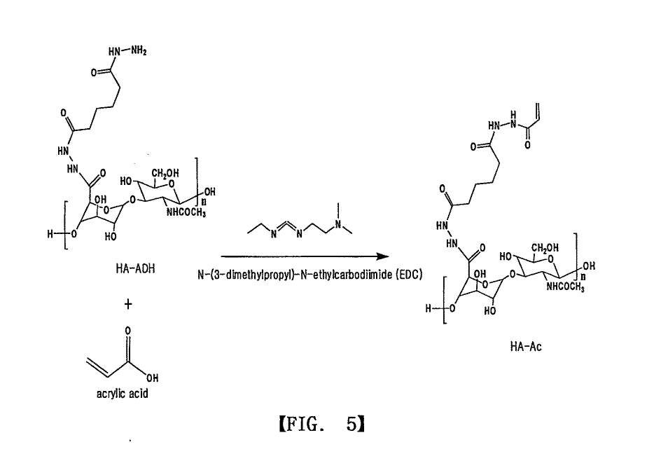

Hyaluronic acid used for preparing the hydrogel and microbeads according to the present invention is an acrylate- or methacrylate-containing hyaluronic acid derivative, formed via crosslinking between the carboxylic acid functional groups of hyaluronic acid and amine functional groups of acrylate or methacrylate. According to a preferred embodiment of the present invention, hyaluronic acid-methacrylate, hyaluronic acid-adipic acid hydrazide tert-butyl hydrazide protected with a tert-butyl group and hyaluronic acid-acrylate compounds are obtained via the reaction schemes as shown in FIGS. 3, 4 and 5.

More particularly, as a chitosan derivative for preparing a hydrogel, chitosan-amidoacrylate is prepared by chemically combining methacrylic acid with chitosan, or chitosan-2-carboethyl acrylate is prepared by chemically combining 2-carboxyethyl acrylate with chitosan.

Also, as a hyaluronic acid derivative for preparing a hydrogel, hyaluron-amide propyl methacrylate is prepared by chemically combining aminopropyl methacrylate with hyaluronic acid, or hyaluronic acid-hydrazide adipic acid hydrazide acrylate is prepared by chemically combining mono-tert-butyl hydrazide adipic acid hydrazide acrylate with hyaluronic acid.

The acrylate- or methacrylate-containing substance that can be crosslinked with chitosan includes, but is not limited to: acrylic acid, methacrylic acid, acrylamide, methacrylamide, alkyl-(meth)acrylamide, mono-tert-Butyl hydrazide adipic acid hydrazide acrylate, N-mono-(meth)acrylamide, N,N-di-C₁-C₄ alkyl-(meth)acrylamide, N-butyl(meth)acrylate, methyl(meth)acrylate, ethyl(meth)acrylate, isobornyl (meth)acrylate, cyclohexyl(meth)acrylate, hydroxyethylacrylate, hydroxyethyl methacrylate, hydroxypropyl acrylate, hydroxypropyl methacrylate, hydroxybutyl acrylate, N-(2-hydroxyethyl)acrylamide, N-methyl acrylamide, N-butoxymethyl acrylamide, N-methoxymethylacrylamide, N-methoxy methylmethacrylamide, 2-acrylamidoglycolic acid, 2-carboxyethyl acrylate, or the like.

The chitosan derivative and the hyaluronic acid derivative are allowed to form covalent bonds with a thiol functional group-containing substance to provide the chitosan-hyaluronic acid-polyethylene oxide hydrogel and hydrogel microbeads according to the present invention. Herein, acrylate and/or methacrylate functional groups and the thiol functional groups are used in a ratio of 8:1˜1:8, and the ratio may be controlled to induce cell adhesion or anti-adhesion. Preferably, the ratio of the acrylate and/or methacrylate functional groups to the thiol functional groups is 3:1˜1:2, more preferably 1:1.

The chitosan derivative and the hyaluronic acid derivative may be mixed in various ratios to provide the chitosan-hyaluronic acid-polyethylene oxide hydrogel and hydrogel microbeads. Herein, the ratio of chitosan to hyaluronic acid may be selected in a broad range of 99:1˜1:99 to optimize biological and mechanical properties of chitosan or hyaluronic acid while optimizing and controlling the time required for preparing hydrogels. Also, the ratio of acrylate to methacrylate bound to chitosan or hyaluronic acid may be selected in a broad range of 100:0˜0:100 to control the time required for preparing the hydrogel and hydrogel microbeads.

The thiol functional group-containing substance combined with the chitosan derivative or the hyaluronic acid derivative includes polyethylene oxide, polypropylene oxide, allyl glycidyl ether, or the like, but is not limited thereto. More preferably, the thiol functional group-containing substance is polyethylene oxide, and the ratio of the chitosan derivative or the hyaluronic acid derivative to polyethylene oxide may be controlled to obtain a hydrogel for controlling anti-adhesion of cells.

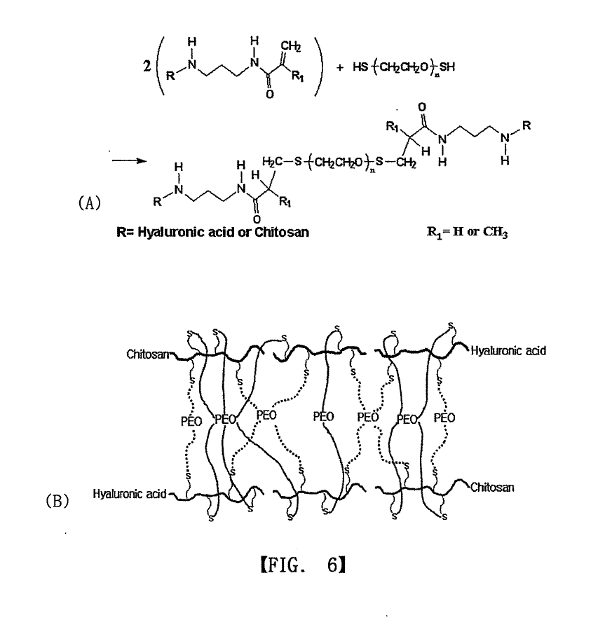

Particularly, a chitosan-hyaluronic acid-polyethylene oxide hydrogel (FIG. 4) and hydrogel microbeads are prepared via reactions between thiol groups of thiol functional group-containing polyethylene oxide and acrylate and/or methacrylate functional groups of chitosan-acrylate, chitosan-methacrylate, hyaluronic acid-acrylate, hyaluronic acid-methacrylate and a mixture of thereof.

The chitosan-chitosan-polyethylene oxide hydrogel and hydrogel microbeads, the hyaluronic acid-hyaluronic acid-polyethylene oxide hydrogel and hydrogel microbeads, and chitosan-hyaluronic acid-polyethylene oxide hydrogel and hydrogel microbeads may be used in various applications including a wound-healing patch, a plastic surgical material, cosmetic material or a scaffold for tissue engineering. Additionally, the chitosan-chitosan-polyethylene oxide hydrogel, the hyaluronic acid-hyaluronic acid-polyethylene oxide hydrogel and the chitosan-hyaluronic acid-polyethylene oxide hydrogel may be used as a bioactive substance delivery carrier. Since polyethylene oxide, chitosan and hyaluronic acid are known as substances having biocompatibility, their use in a bioactive substance delivery carrier is more preferred.

Also, the present invention provides a bioactive substance delivery carrier comprising a bioactive substance supported on the chitosan-chitosan-polyethylene oxide, hyaluronic acid-hyaluronic acid-polyethylene oxide and chitosan-hyaluronic acid-polyethylene oxide hydrogel and hydrogel microbeads.

As used herein, the term “bioactive substance” means a substance for use in treating, healing, preventing or diagnosing diseases and is not limited to a specific substance or species. Such bioactive molecules include organic compounds, extract, proteins, peptides, PNA (peptide nucleic acid), lipid, carbohydrates, steroids, extracellular matrix substances, cells, or the like. Additionally, various excipients currently used in the art, such as a diluent, a release controlling agent, inert oil or a binder, may be mixed with the bioactive substance.

As used herein, the term “bioactive substance delivery carrier” means a system on which a bioactive substance is supported for the purpose of in vivo delivery. According to the present invention, a bioactive substance is supported on the chitosan-chitosan-polyethylene oxide hydrogel and hydrogel microbeads, the hyaluronic acid-hyaluronic acid-polyethylene oxide hydrogel and hydrogel microbeads, chitosan-hyaluronic acid-polyethylene oxide hydrogel and hydrogel microbeads, and a chitosan-hyaluronic acid-polyethylene oxide-protein or chitosan-hyaluronic acid-polyethylene oxide-peptide hydrogel and hydrogel microbeads, so that it can be delivered into the body. As desired, it is also possible to allow a bioactive substance to be released at a predetermined site constantly over a predetermined period of time. Such controlled releasing type carriers have an advantage in that they can control the releasing rates of drugs having such low bioavailability or high absorptivity as to be discharged too fast from the body, and thus can maintain a desired drug concentration in blood for a long period of time. In the chitosan-chitosan-polyethylene oxide hydrogel and hydrogel microbeads, the hyaluronic acid-hyaluronic acid-polyethylene oxide hydrogel and hydrogel microbeads, chitosan-hyaluronic acid-polyethylene oxide hydrogel and hydrogel microbeads, degradability of the gels and releasing rates of bioactive substances may be controlled depending on the physical strength and chemical properties of the gels.

Organic compounds that may be supported on the chitosan-chitosan-polyethylene oxide hydrogel and hydrogel microbeads, the hyaluronic acid-hyaluronic acid-polyethylene oxide hydrogel and hydrogel microbeads, chitosan-hyaluronic acid-polyethylene oxide hydrogel and hydrogel microbeads so as to be delivered into the body include conventional antibiotics, anti-cancer agents, anti-inflammatory agents, anti-viral agents, antibacterial agents, or the like. Particular examples of antibiotics include an antibiotic selected from the group consisting of tetracycline, minocycline, doxycycline, ofloxacin, revofloxacin, ciprofloxacin, clarithromycin, erythromycin, cefaclor, cefotaxim, imipenem, penicillin, gentamycin, streptomycin, bancomycin, or a derivative or mixture thereof. Particular examples of anti-cancer agents include methotrexate, carboplatin, taxol, cisplatin, 5-fluorouracil, doxorubicin, etpocide, paclitaxel, camtotecin, cytosine, arabinose, and derivatives and mixtures thereof. Particular examples of anti-inflammatory agents include an anti-inflammatory agent selected from the group consisting of indometacin, ibuprofen, ketoprofen, piroxicam, flubiprofen, diclofenac, and derivatives and mixtures thereof. Particular examples of anti-viral agents include an anti-viral agent selected from the group consisting of acyclovir, robavin, and derivatives and mixtures thereof. Particular examples of antibacterial agents include an antibacterial agent selected from the group consisting of ketoconazole, itraconazole, fluconazole, amphotericin-B, griceofulvin, and derivatives and mixtures thereof.

Proteins and peptides that may be supported on the chitosan-chitosan-polyethylene oxide hydrogel and hydrogel microbeads, the hyaluronic acid-hyaluronic acid-polyethylene oxide hydrogel and hydrogel microbeads, chitosan-hyaluronic acid-polyethylene oxide hydrogel and hydrogel microbeads so as to be delivered into the body include various bioactive peptides for use in treating and preventing diseases, such as, hormones, cytokines, enzymes, antibodies, growth factors, transcription control factors, blood factors, vaccines, structural proteins, ligand proteins and receptors, cell surface antigens, and derivatives and analogues thereof.

Particular examples of the proteins and peptides include: liver growth hormone, growth hormone-releasing hormone, growth hormone-releasing peptide, interferon and interferon receptors (e.g. interferon-alpha, -beta and -gamma, aqueous type I interferon receptor, etc.), granulocyte colony stimulating factor (G-CSF), granulocyte-macrophage colony stimulating factor (GM-SCF), glucagon-like peptides (e.g. GLP-1), G-protein-coupled receptor, interleukines (e.g. interleukine-1, -2, -3, -4, -5, -6, -7, -8, -9, etc.), interleukine receptors (e.g. IL-1 receptor, IL-4 receptor, etc.), enzymes (e.g. glucocerebrosidase, iduronate-2-sulfatase, alpha-galactosidase-A, agalsidase-alpha, agalsidase -beta, alpha-L-iduronidase, butyrylcholine stearase, chitinase, glutamate dicarboxylase, imiglucerase, lipase, uricase, platelet-activating factor acetylhydrolase, neutral endopeptidase, myeloperoxidase, etc.), interleukine- and cytokine-binding proteins (e.g. IL-18 bp, TNF-binding protein, etc.), macrophage-activating factor, macrophage peptides, B-cell factor, T-cell factor, protein A, allergy inhibiting factor, apoptosis glycoprotein, immunotoxin, limphotoxin, tumor necrosis factor, tumor inhibiting factor, transforming growth factor, alpha-1 antitrypsin, albumin, alpha-lactalbumin, apolipoprotein-E, erythropoietin, high-saccharide chain erythropoietin, angiopoietin, hemoglobin, thrombin, thrombin receptor activating peptide, thrombomodulin, blood factor VII, blood factor VIIa, blood factor VIII, blood factor IX, blood factor XIII, plasminogen activating factor, fibrin-binding peptide, eurokinase, streptokinase, hirudin, protein C, C-reactive protein, rennin inhibitor, colagenase inhibitor, superoxide dismutase, leptin, platelet-derived growth factor, epithelial cell growth factor, epidermal cell growth factor, angiostatin, angiotensin, bone morphogenetic growth factor, bone morphogenetic protein, calcitonin, insulin, atriopeptin, cartilage-inducing factor, elcatonin, connective tissue activating factor, tissue factor pathway inhibitor, follicle-stimulating hormone, lutenizing hormone, lutenizing hormone-releasing hormone, nerve growth factors (e.g. neurotrophin, cilliary neurotrophic factor, axogenesis factor-1, brain-natriuretic peptide, glial-derived neurotrophic factor, netrin, neutrophil inhibitory factor, neurotrophic factor, neutrin, etc.), parathyroid hormone, relaxin, secretin, somatomedin, insulin-like growth factor, adrenal cortex hormone, glucagone, cholecystokinine, pancreatic polypeptide, gastrin-releasing peptide, corticotropine-releasing factor, thyroid stimulating hormone, autotaxin, lactoferrin, myostatin, receptors (e.g. TNFR(P75), TNFR(P55), IL-1 receptor, VEGF receptor, B-cell activating factor receptor, etc.), receptor antagonists (e.g. IL1-Ra, etc.), cell surface antigens (e.g. CD 2, 3, 4, 5, 7, 11a, 11b, 18, 19, 20, 23, 25, 33, 38, 40, 45, 69, etc.), monoclonal antibody, polyclonal antibody, antibody fragments (e.g. scFv, Fab, Fab′, F(ab′)₂, Fd, etc.), virus-derived vaccine antigen, or the like.

Nucleic acids that may be supported on the chitosan-chitosan-polyethylene oxide hydrogel and hydrogel microbeads, the hyaluronic acid-hyaluronic acid-polyethylene oxide hydrogel and hydrogel microbeads, chitosan-hyaluronic acid-polyethylene oxide hydrogel and hydrogel microbeads so as to be delivered into the body include DNA, RNA, oligonucleotides, or the like.

Extracellular matrix substances that may be supported on the chitosan-chitosan-polyethylene oxide hydrogel and hydrogel microbeads, the hyaluronic acid-hyaluronic acid-polyethylene oxide hydrogel and hydrogel microbeads, chitosan-hyaluronic acid-polyethylene oxide hydrogel and hydrogel microbeads so as to be delivered into the body include collagen, fibronectin, gelatin, laminin, vitronectin, or the like. Cells that may be used in the present invention include fibroblasts, vascular endothelial cells, smooth muscle cells, nerve cells, chondrocytes, bone cells, dermal cells, Schwann cells, stem cells, or the like.

In fact, after smooth muscle cells were cultured on the surface of the hydrogel according to the present invention, it could be seen that there was an increase in cell count in 3 days. Also, when using the hydrogel as a cell delivery carrier, proliferation of the cells supported on the hydrogel and an increase in cell count were observed. Further, after about two weeks to several months, the hydrogel was degraded and the cells were attached to the surface of a cell culture flask. This indicates that it is possible to obtain stable maintenance and activity of a bioactive substance by supporting the substance on the chitosan-hyaluronic acid-polyethylene oxide hydrogel according to the present invention.

Further, the present invention provides a chitosan-hyaluronic acid-polyethylene oxide-peptide hydrogel and hydrogel microbeads, which is obtained by combining the chitosan derivative and the hyaluronic acid derivative in the chitosan-chitosan-polyethylene oxide, hyaluronic acid-hyaluronic acid-polyethylene oxide and chitosan-hyaluronic acid-polyethylene oxide hydrogel and hydrogel microbeads with thiol functional group-containing substances comprising a cysteine amino acid-containing peptide or fibronectin-containing protein in addition to polyethylene oxide. The chitosan-hyaluronic acid-polyethylene oxide-peptide hydrogel and hydrogel microbeads may be used as a scaffold for tissue engineering. The cysteine amino acid-containing peptide refers to a peptide having an amino acid sequence capable of inducing cell adhesion and/or cell migration and proliferation and cysteine amino acid for carrying out crosslinking with (meth)acrylate chitosan/(meth)acrylate hyaluronic acid/polyethylene oxide (for example, GSRGDSC), a peptide containing an amino acid sequence (for example, YKNR) having controlled biodegradability due to enzymes, such as collagenase or plasmin, and cysteine, or other peptides having a function different from the above peptides.

As used herein, the term “scaffold for tissue engineering” means a hydrogel and hydrogel microbeads comprising a chitosan-chitosan-polyethylene oxide-peptide and chitosan-hyaluronic acid-polyethylene oxide-peptide obtained by chemically combining a peptide having a function of inducing tissue regeneration with the chitosan-chitosan-polyethylene oxide, hyaluronic acid-hyaluronic acid-polyethylene oxide and chitosan-hyaluronic acid-polyethylene oxide hydrogel and hydrogel microbeads. The peptide refers to an oligopeptide or protein containing cysteine as an amino acid, and the thiol functional groups contained in cysteine reacts and is chemically crosslinked with (meth)acrylate functional groups to form a chitosan-chitosan-polyethylene oxide-peptide hydrogel, hyaluronic acid-hyaluronic acid-polyethylene oxide-peptide hydrogel and chitosan-hyaluronic acid-polyethylene oxide-peptide hydrogel and hydrogel microbeads. The amino acid sequence contained in the peptide serves to provide a site for cell adhesion, cell proliferation (e.g. RGD) or for enzymatic degradation of a scaffold (e.g. YKNR) to induce tissue regeneration. The peptide provides a site that allows focal contact or cell adhesion of the cells contained in the hydrogel or gel. Additionally, the site for the degradation of a scaffold induces degradation of the hydrogel, so that the cells adhered to the scaffold are degraded according to the degradation of the scaffold, resulting in cell migration and proliferation. Finally, the hydrogel is degraded and removed, and the space occupied originally by the hydrogel is substituted with newly regenerated tissue formed by an extracellular matrix secreted by the cells and such proliferated cells.

Particular examples of the peptide that may be used in the chitosan-chitosan-polyethylene oxide-peptide hydrogel, hyaluronic acid-hyaluronic acid-polyethylene oxide-peptide hydrogel and chitosan-hyaluronic acid-polyethylene oxide-peptide hydrogel include: oligopeptides such as RGD, RGDS, REDV and YIGSR capable of cell adhesion; cysteine-containing extracellular matrix substances such as collagen, fibronectin, gelatin, elastin, osteocalcin, fibrinogen, fibromodulin, tenascin, laminin, osteopontin, osteonectin, perlecan, versican, von Willebrand factor and vitronectin; organic compounds degraded by a specific enzyme, such as YKNR; or the like. Herein, RGE, REDV, YKNR, etc., are expressed by single-letter abbreviation of amino acids.

Further, the present invention provides a method for preparing a chitosan-chitosan-polyethylene oxide hydrogel, the method comprising the steps of: (i) providing an aqueous chitosan solution; (ii) crosslinking chitosan with an acrylate functional group-containing substance to provide a chitosan derivative; (iii) crosslinking chitosan with a methacrylate functional group-containing substance to provide a chitosan derivative; and (iv) forming covalent bonds between a mixture of the chitosan derivatives and a thiol functional group-containing substance.

Further, the present invention provides a method for preparing a hyaluronic acid-hyaluronic acid-polyethylene oxide hydrogel, the method comprising the steps of: (i) providing an aqueous hyaluronic acid solution; (ii) crosslinking hyaluronic acid with an acrylate functional group-containing substance to provide a hyaluronic acid derivative; (iii) crosslinking hyaluronic acid with a methacrylate functional group-containing substance to provide a hyaluronic acid derivative; and (iv) forming covalent bonds between a mixture of the hyaluronic acid derivatives and a thiol functional group-containing substance.

Further, the present invention provides a method for preparing a chitosan-hyaluronic acid-polyethylene oxide hydrogel, the method comprising the steps of: (i) providing an aqueous chitosan solution and an aqueous hyaluronic acid solution; (ii) crosslinking chitosan with an acrylate or methacrylate functional group-containing substance to provide a chitosan derivative; (iii) crosslinking hyaluronic acid with an acrylate or methacrylate functional group-containing substance to provide a hyaluronic acid derivative; and (iv) forming covalent bonds between the chitosan derivative as well as the hyaluronic acid derivative and a thiol functional group-containing substance.

In step (i), chitosan and hyaluronic acid may be dissolved in water or an acidic solution.

In steps (ii) and (iii), the acrylate- or methacrylate-containing substance may be crosslinked with chitosan or hyaluronic acid by using a crosslinking agent. Particular examples of the crosslinking agent that may be used in the present invention include ethylene glycol, glycerin, polyoxyethylene glycol, bisacryl amide, diaryl phthalate, diaryl adipate, 1,4-butanediol diglycidyl ether, polyethylene glycol diglycidyl ether, polypropylene glycol diglycidyl ether, triglycerine diglycidyl ether, triaryl amine, glyoxal, diethyl propyl ethyl carbodiimide hydrochloride, carbodiimide (CDI), or the like.

In a preferred embodiment of the present invention, diethylpropylethyl carbodiimide hydrochloride (EDC) is used as a crosslinking agent. It is possible to control the molar ratio of chitosan:2-acrylamido glycolic acid:EDC and that of hyaluronic acid:adipic dihydrazide: acrylic acid: EDC in a broad range. In fact, when preparing a chitosan hydrogel, the above molar ratio can be varied diversely, for example 1:4:4, 1:8:8 or 1:12:8 to form the hydrogel.

In step (iv), the ratio of acrylate or methacrylate functional groups to thiol functional groups may be controlled as necessary. The ratio of acrylate or methacrylate functional groups to thiol functional groups may be 4:1 to 1:3. Preferably, the ratio is 3:1 to 1:2, more preferably 1:1.

The resultant hydrogel may have different levels of physical strength and chemical properties according to various factors, including the molecular weights of chitosan and hyaluronic acid used for preparing the hydrogel, particular type of the molecule containing acrylate or methacrylate functional groups, concentrations and deacetylation degrees of chitosan and hyaluronic acid, particular type and concentration of the crosslinking agent used for preparing the hydrogel, pH, or the ratio of acrylate or methacrylate functional groups to thiol functional groups in the reaction mixture. A desired hydrogel can be prepared considering all of the above factors. For example, water content of a gel may be varied depending on the molar ratio of chitosan:2-acrylamido glycolic acid: EDC and the number of thiol groups bound to PEO. In the case of hyaluronic acid, it is possible to control the properties of the gel formed from hyaluronic acid depending on the molar ratio of hyaluronic acid:aminopropyl methacrylate:EDC and the number of thiol groups bound to PEO.

More particularly, a method for preparing the chitosan-hyaluronic acid-polyethylene oxide hydrogel comprises the steps of: providing an aqueous chitosan solution and an aqueous hyaluronic acid solution; crosslinking chitosan with an acrylate-containing substance to provide a chitosan derivative; crosslinking hyaluronic acid with a methacrylate-containing substance to provide a hyaluronic acid derivative; removing unreacted acrylate- and methacrylate-containing reactants from the chitosan derivative and the hyaluronic acid derivative; drying the chitosan derivative and the hyaluronic acid derivative; and forming covalent bonds between the chitosan derivative as well as the hyaluronic acid derivative and a thiol functional group-containing substance.

Further, the present invention provides a method for preparing a bioactive substance delivery carrier, the method comprising the steps of: (i) providing an aqueous chitosan solution; (ii) crosslinking chitosan with an acrylate functional group-containing substance to provide a chitosan derivative; (iii) crosslinking chitosan with a methacrylate functional group-containing substance to provide a chitosan derivative; (iv) mixing a bioactive substance with the chitosan derivatives or a thiol functional group-containing substance; and (v) forming covalent bonds between the chitosan derivatives and the thiol functional group-containing substance while the bioactive substance is supported thereon.

Further, the present invention provides a method for preparing a bioactive substance delivery carrier, the method comprising the steps of: (i) providing an aqueous hyaluronic acid solution; (ii) crosslinking hyaluronic acid with an acrylate functional group-containing substance to provide a hyaluronic acid derivative; (iii) crosslinking hyaluronic acid with a methacrylate functional group-containing substance to provide a hyaluronic acid derivative; (iv) mixing a bioactive substance with the hyaluronic acid derivatives or a thiol functional group-containing substance; and (v) forming covalent bonds between the hyaluronic acid derivatives and the thiol functional group-containing substance while the bioactive substance is supported thereon.

Further, the present invention provides a method for preparing a bioactive substance delivery carrier, the method comprising the steps of: (i) providing an aqueous chitosan solution and an aqueous hyaluronic acid solution; (ii) crosslinking chitosan with an acrylate or methacrylate functional group-containing substance to provide a chitosan derivative; (iii) crosslinking hyaluronic acid with an acrylate or methacrylate functional group-containing substance to provide a hyaluronic acid derivative; (iv) mixing a bioactive substance with the chitosan derivative and the hyaluronic acid derivative, or with a thiol functional group-containing substance; and (v) forming covalent bonds between the chitosan derivative as well as the hyaluronic acid derivative and the thiol functional group-containing substance while the bioactive substance is supported thereon.

According to the present invention, a step of supporting a bioactive substance on the chitosan-chitosan-polyethylene oxide hydrogel, the hyaluronic acid-hyaluronic acid-polyethylene oxide hydrogel and the chitosan-hyaluronic acid-polyethylene oxide hydrogel may be carried out during the preparation of the gel, or after preparing the gel for the subsequent use. However, step (iv) is preferably performed by supporting a bioactive substance on the gel during the preparation of the gel, more particularly, by incorporating a bioactive substance into the chitosan derivative solution, the hyaluronic acid derivative solution or a mixed solution of the chitosan derivative with the hyaluronic acid derivative, obtained from steps (ii) and (iii). The bioactive substance is mixed with the chitosan derivative solution, the hyaluronic acid derivative solution or the solution containing the thiol functional group-containing substance dissolved therein, so that the substance can form covalent bonds with the gel.

More particularly, the method for preparing a hydrogel as a bioactive substance delivery carrier comprises the steps of: providing an aqueous chitosan solution and an aqueous hyaluronic acid solution; crosslinking chitosan with an acrylate-containing substance to provide a chitosan derivative; crosslinking hyaluronic acid with a methacrylate-containing substance to provide a hyaluronic acid derivative; removing unreacted acrylate- and methacrylate-containing reactants from the chitosan derivative and the hyaluronic acid derivative; drying the chitosan derivative and the hyaluronic acid derivative; mixing a bioactive substance with the chitosan derivative and the hyaluronic acid derivative, or with a thiol functional group-containing substance; and forming covalent bonds between the chitosan derivative as well as the hyaluronic acid derivative and the thiol functional group-containing substance.

Further, the present invention provides a method for preparing hydrogel microbeads as a bioactive substance delivery carrier, the method comprising the steps of: (i) providing an aqueous chitosan solution and an aqueous hyaluronic acid solution; (ii) crosslinking chitosan with an acrylate or methacrylate functional group-containing substance to provide a chitosan derivative; (iii) crosslinking hyaluronic acid with an acrylate or methacrylate functional group-containing substance to provide a hyaluronic acid derivative; (iv) mixing a bioactive substance with the chitosan derivative and the hyaluronic acid derivative, or with a thiol functional group-containing substance; (v) mixing the chitosan derivative and the hyaluronic acid derivative with the thiol functional group-containing substance to provide a mixed solution while the bioactive substance is supported thereon; (vi) adding the mixed solution dropwise to a solution containing a hydrophobic solvent and a surfactant and dispersing the mixed solution therein; and (vii) allowing the chitosan derivative, the hyaluronic acid derivative and polyethylene oxide dispersed in the solution to form hydrogel microbeads and recovering the microbeads.

Reference will now be made in detail to the preferred embodiments of the present invention. However, the following examples and comparative examples are illustrative only, and the scope of the present invention is not limited thereto.

### Example 1

### Preparation of Chitosan Methacrylate Derivative Hydrogel Containing Bioactive Element

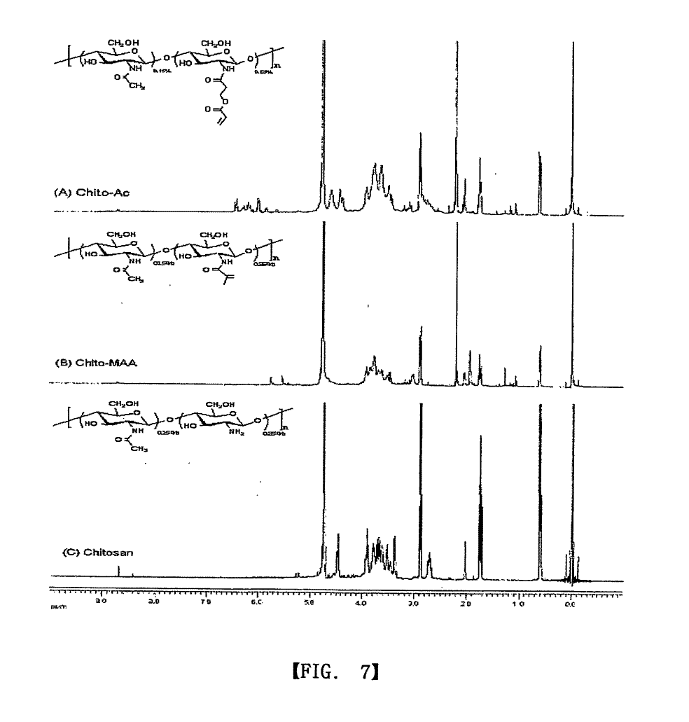

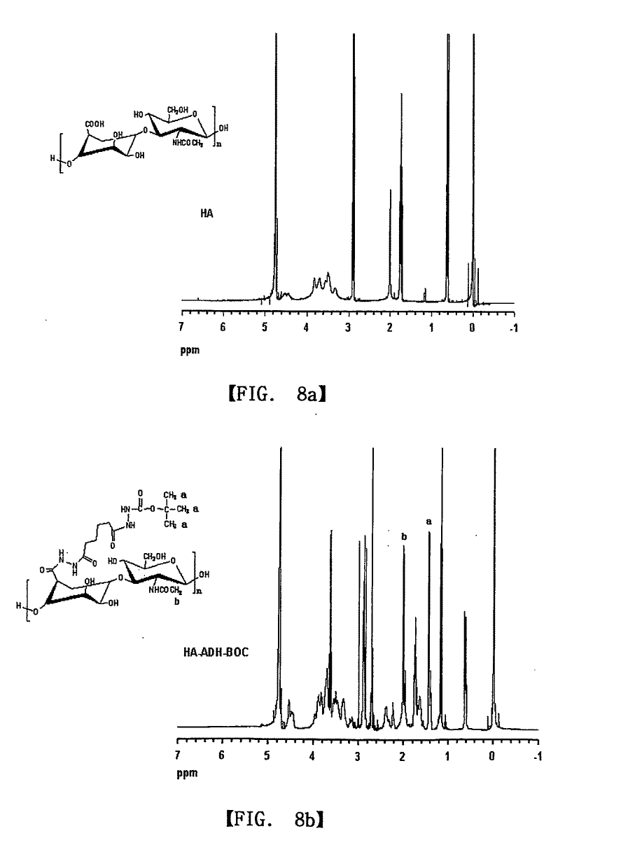

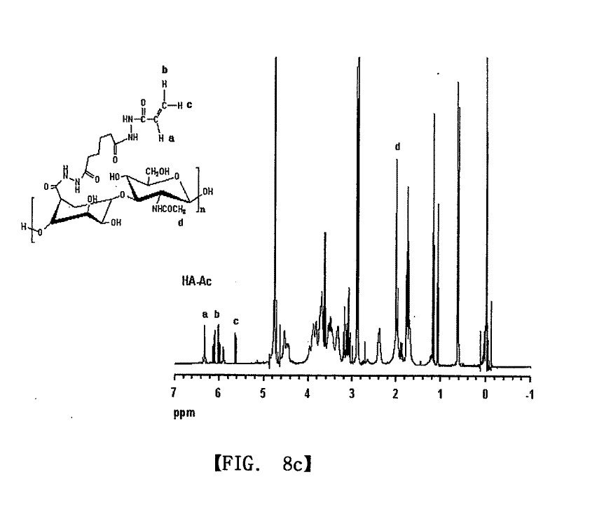

Step 1: 20 mL of aqueous chitosan (5˜10 KDa; Chitolife, Korea) having a deacetylation degree of about 85% was mixed with 0.3 mL of methacrylic acid, and 5 mL of EDC was added thereto to perform reaction while stirring the reaction mixture. After the completion of the reaction, the resultant product was precipitated by using an organic solvent, and was freeze-dried for one day to obtain a first product of chitosan-methacrylate under a molar ratio of 1 (chitosan):4 (2-carboxyethyl acrylic acid):4 (EDC) (see FIG. 7-B).

Step 2: The chitosan-methacrylate obtained from Step 1 was dissolved in triethanol amine to provide 0.1 mL of chitosan-methacrylate solution. In a separate container, a polyethylene oxide polymer having six arms of thiol functional groups was dissolved in triethanol amine to provide 0.1 mL of polyethylene oxide solution.

Step 3: The above two solutions were mixed with each other. At this time, it could be observed by the naked eyes that a chitosan methacrylate-polyethylene oxide hydrogel was formed over a period of 24˜30 hours.

### Example 2

Chitosan-2-carboxyethyl acrylate was prepared in the same manner as described in Example 1, except that 2-carboxyethyl acrylate was used instead of methacrylic acid, and the resultant product was evaluated by NMR (see FIG. 7-A).

### Example 3

Chitosan-2-acrylamidoglycolic acid was prepared in the same manner as described in Example 1, except that 2-acrylamido glycolic acid monohydrate was used instead of methacrylic acid. The chitosan-2-acrylamido glycolic acid was allowed to react with polyethylene oxide in the same manner as described in Example 1. After the reaction, a chitosan acrylate-polyethylene oxide hydrogel was obtained within 2 minutes.

### Example 4

Hyaluronic acid-N-(3-aminopropyl)methacrylamide was prepared in the same manner as described in Example 1, except that hyaluronic acid (MW 10 k˜100 k) was used instead of the aqueous chitosan and N-(3-aminopropyl)methacrylamide (APM) was used instead of methacrylic acid. The resultant product, hyaluronic acid-N-3-aminopropyl methacrylamide was allowed to react with polyethylene oxide to obtain a hyaluronic acid methacrylate-polyethylene oxide hydrogel within 24 hours.

### Example 5

The chitosan-methacrylate obtained from Example 1 was mixed with the chitosan-2-carboxyethyl acrylate obtained from Example 2 in a ratio of 25%:75%, and the resultant mixed solution was allowed to react with polyethylene oxide in the same manner as described in Example 1 to obtain a chitosan acrylate-chitosan methacrylate-polyethylene oxide hydrogel within 2 hours.

### Example 6

The chitosan-methacrylate obtained from Example 1 was mixed with the chitosan-2-carboxyethyl acrylate obtained from Example 2 in a ratio of 50%:50%, and the resultant mixed solution was allowed to react with polyethylene oxide in the same manner as described in Example 1 to obtain a chitosan acrylate-chitosan methacrylate-polyethylene oxide hydrogel within 4 hours.

### Example 7

The chitosan-methacrylate obtained from Example 1 was mixed with the chitosan-2-carboxyethyl acrylate obtained from Example 2 in a ratio of 75%:25%, and the resultant mixed solution was allowed to react with polyethylene oxide in the same manner as described in Example 1 to obtain a chitosan acrylate-chitosan methacrylate-polyethylene oxide hydrogel within 5 hours.

### Example 8

The chitosan-2-carboxyethyl acrylate obtained from Example 3 was mixed with the hyaluronic acid-N-(3-aminopropyl)methacrylamide obtained from Example 4 in a ratio of 75%:25%, and the resultant mixed solution was allowed to react with polyethylene oxide in the same manner as described in Example 1 to obtain a chitosan acrylate-hyaluronic acid methacrylate-polyethylene oxide hydrogel within 2 hours.

### Example 9

The chitosan-2-carboxyethyl acrylate obtained from Example 3 was mixed with the hyaluronic acid-N-(3-aminopropyl)methacrylamide obtained from Example 4 in a ratio of 50%:50%, and the resultant mixed solution was allowed to react with polyethylene oxide in the same manner as described in Example 1 to obtain a chitosan acrylate-hyaluronic acid methacrylate-polyethylene oxide hydrogel within 4 hours.

### Example 10

The chitosan-2-carboxyethyl acrylate obtained from Example 3 was mixed with the hyaluronic acid-N-(3-aminopropyl)methacrylamide obtained from Example 4 in a ratio of 25%:75%, and the resultant mixed solution was allowed to react with polyethylene oxide in the same manner as described in Example 1 to obtain a chitosan acrylate-hyaluronic acid methacrylate-polyethylene oxide hydrogel within 5 hours.

### Example 11

The chitosan-methacrylate obtained from Example 1 was mixed with the chitosan-acrylate obtained from Example 2. When mixing the above chitosan derivatives, a solution containing collagen dissolved in acetic acid (0.1% (w/w) or 0.3% (w/w) based on the weight of the chitosan-(meth)acrylate) was further added thereto, so as to obtain a chitosan acrylate-chitosan methacrylate-polyethylene oxide hydrogel containing 0.1% or 0.3% of collagen added thereto.

### Example 12

In Step 2 of preparing a hydrogel in Example 1, a solution containing collagen dissolved in acetic acid (0.1% (w/w) or 0.3% (w/w) based on the weight of the chitosan-(meth)acrylate) was further mixed with the solutions, so as to obtain a chitosan acrylate-polyethylene oxide hydrogel or chitosan methacrylate-polyethylene oxide hydrogel containing 0.1% or 0.3% of collagen added thereto.

### Example 13

In Step 2 of preparing a hydrogel in Example 1, a solution containing fibronectin dissolved in ultra-pure water (0.1% (w/w) or 0.3% (w/w) based on the weight of the chitosan-(meth)acrylate) was further mixed with the solutions, so as to obtain a chitosan acrylate-polyethylene oxide hydrogel or chitosan-methacrylate-polyethylene oxide hydrogel containing 0.1% or 0.3% of fibronectin added thereto.

### Example 14

When the chitosan-methacrylate obtained from Example 1 was mixed with the chitosan-acrylate obtained from Example 3, a solution containing fibronectin dissolved in ultra-pure water (0.1% (w/w) or 0.3% (w/w) based on the weight of the chitosan-(meth)acrylate) was further mixed with the solutions, so as to obtain a chitosan acrylate-polyethylene oxide hydrogel or chitosan methacrylate-polyethylene oxide hydrogel containing 0.1% or 0.3% of fibronectin added thereto.

### Example 15

When preparing a hydrogel in Example 9, a chitosan solution containing fibronectin dissolved in ultra-pure water (0.3% (w/w) based on the weight of the chitosan-(meth)acrylate) was mixed with the hyaluronic acid-methacrylate solution, so as to obtain a chitosan acrylate-hyaluronic acid methacrylate-polyethylene oxide hydrogel containing 0.3% of fibronectin added thereto.

### Example 16

When preparing a hydrogel, 5 μL or 15 μL of a solution containing collagen dissolved in sterilized distilled water in an amount of 0.1% or 0.3% based on the combined weight of the chitosan-methacrylate and the chitosan-acrylate obtained from Step 2 in Examples 1 and 3, and 10 μL of a cell suspension containing 5,000 smooth muscle cells were provided individually in a 1.5 mL micro-conical tube. In a separate 1.5 mL micro-conical tube, 300 μL of thiol group-containing polyethylene oxide-triethanol amine solution and 300 μL of mixed chitosan-acrylate and chitosan-methacrylate-triethanol amine solution were provided individually. To the collagen solution, the suspension of smooth muscle cells was incorporated to provide a cell-collagen solution, which, in turn, was mixed with the polyethylene oxide solution. The resultant cell-collagen-polyethylene oxide solution was mixed with the solution of chitosan-acrylate and chitosan-methacrylate to provide a chitosan methacrylate-chitosan acrylate-polyethylene oxide-collagen hydrogel containing the cells.

### Example 17

Example 16 was repeated to provide a chitosan methacrylate-chitosan acrylate-polyethylene oxide-fibronectin hydrogel containing cells, except that fibronectin was used instead of collagen.

### Example 18

Example 16 was repeated to provide a chitosan acrylate-hyaluronic acid methacrylate-polyethylene oxide-collagen hydrogel containing cells, except that hyaluronic acid-methacrylate was used instead of chitosan-methacrylate.

### Example 19

In the step of preparing a hydrogel in Example 18, a solution containing fibronectin, instead of collagen, dissolved in triethanol amine (0.3% (w/w) based on the weight of chitosan-acrylate and hyaluronic acid-methacrylate) was mixed with the solutions to provide a chitosan acrylate-hyaluronic acid methacrylate-polyethylene oxide-fibronectin hydrogel containing fibronectin added thereto.

### Example 20

In Step 2 of preparing a hydrogel in Example 18, a solution containing CGRGDGC peptide, instead of collagen, dissolved in triethanol amine (0.3% (w/w) based on the weight of chitosan acrylate-hyaluronic acid methacrylate) was mixed with the solutions to provide a peptide-chitosan acrylate-hyaluronic acid methacrylate-polyethylene oxide hydrogel containing cysteine as an amino acid.

### Example 21

First, one end of adipic acid dihydrazide was protected with tert-butyl group to form tert-butyl adipic acid hydrazide, and the resultant product was chemically combined with hyaluronic acid to provide hyaluronic acid-adipic acid hydrazide. Next, the tert-butyl group was removed from hyaluronic acid-adipic acid hydrazide, and the resultant product was combined with acrylic acid to provide hyaluronic acid-acrylate. Then, hyaluronic acid-acrylate was allowed to react with polyethylene oxide to provide a hyaluronic acid acrylate-polyethylene oxide hydrogel.

Hereinafter, the above process is described in detail.

Step 1—Protection of One End of Adipic Acid Dihydrazide: (1) 3.5 g of adipic acid dihydrazide (MW 174 g/mol) was dissolved in 30 mL of mixed solution of tetrahydrofuran/water (THF/H₂O) to provide an adipic acid hydrazide solution. (2) 2.4 g of di-tert-butyl dicarbonate (BOC₂O) was dissolved in mixed solution of tetrahydrofuran/water (THF/H₂O). (3) 2.3 g of NaHCO₃ corresponding to 2.5 times of the amount of di-tert-butyl dicarbonate was added to the solution of di-tert-butyl dicarbonate. (4) The di-tert-butyl dicarbonate solution and NaHCO₃ solution were added gradually to the adipic acid dihydrazide solution to perform reaction, and the resultant product was subjected to freeze-drying. (5) 50 mL of pure water was added to the product to dissolve it, and adipic acid hydrazide tert-butyl hydrazide, one end of which was protected with tert-butyl group, was obtained. (6) Adipic acid hydrazide, both ends of which were protected with tert-butyl groups, were removed to obtain pure adipic acid hydrazide tert-butyl hydrazide, which, in turn, was subjected to freeze-drying to obtain powder.

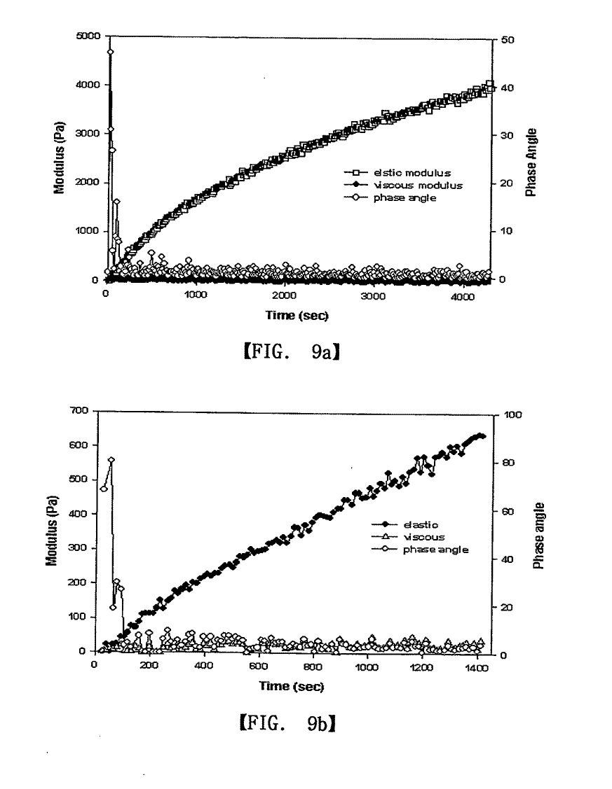

Step 2—Preparation of Hyaluronic Acid-Adipic Acid Hydrazide Compound Whose One End is Protected: (1) 0.68 g (1.7 mmol) of hyaluronic acid and the adipic acid tert-butyl hydrazide (MW=274, 6.8 mmol) protected by using tert-butyl carbonate were dissolved into 40 mL of pure water. (2) 0.9 g (6.8 mmol) of 1-hydroxybenzotriazole hydrate (MW=135) and 1.1 g of EDC (MW=155, 6.8 mmol) were dissolved into 10 mL of mixed solution of dimethyl sulfoxide and pure water (1:1), and the resultant solution was added to a hyaluronic acid solution to perform reaction. (3) The hyaluronic acid solution was added to 500 mL of ethanol to form precipitate and the precipitate was separated. (4) The resultant product was freeze dried for 2 days to obtain tert-butyl adipic acid hydrazide-hyaluronic acid (see FIG. 8-b).

Step 3—Deprotection of Amine Protecting Group: (1) 0.6 g (MW=632 g/mol, 0.95 mmol) of tert-butyl adipic acid hydrazide-hyaluronic acid was dissolved into 6 mL (10%) of distilled water. (2) A mixed solution of hydrogen chloride/methanol (1:1) was added gradually to the solution of tert-butyl adipic acid hydrazide-hyaluronic acid to perform reaction at room temperature for 1˜2 hours. (3) The resultant product was washed with 100 mL of ethanol and freeze-dried to obtain a sample.

Step 4—Preparation of Hyaluronic Acid-Adipic Acid-Acrylate: 0.6 g (MW=532 g/mol) of hyaluronic acid-adipic acid and 0.3 g (MW=72 g/mol, 4 mmol) of acrylic acid were dissolved into 40 mL of distilled water. (2) 0.7 g (MW=155) of EDC was added thereto to perform reaction. (3) The resultant product was precipitated and freeze-dried to obtain a hyaluronic acid-acrylate sample (see FIG. 8-c).

Step 5—Preparation of Hyaluronic Acid Acrylate-Polyethylene Oxide Hydrogel: (1) Hyaluronic acid-adipic acid-acrylate (also referred to hyaluronic acid acrylate hereinafter) was dissolved into triethanol amine buffer to provide 10% (w/v) hyaluronic acid-acrylate solution. (2) Polyethylene oxide having six arms of thiol functional groups was dissolved into triethanol amine buffer to provide 20% (w/v) polyethylene oxide solution. (3) After mixing the above two solutions, hydrogel formation started within 2˜3 minutes, so as to obtain a transparent hyaluronic acid acrylate-polyethylene oxide hydrogel.

### Example 22

Example 9 was repeated, except that hyaluronic acid-acrylate obtained from Example 21, instead of chitosan-2-carboxyethyl acrylate, was mixed with hyaluronic acid-N-(3-aminopropyl)methacrylamide of Example 9 in a ratio of 50:50 to provide a mixed solution. Then, the mixed solution was further mixed with a polyethylene oxide solution to provide a hyaluronic acid acrylate-hyaluronic acid methacrylate-polyethylene oxide hydrogel within one hour.

### Example 23

Hyaluronic acid-acrylate obtained from Example 21 was mixed with chitosan-methacrylate of Example 1 in a ratio of 50:50 to provide a mixed solution. Then, the mixed solution was further mixed with a polyethylene oxide solution. As a result, it could be seen that a hyaluronic acid acrylate-chitosan methacrylate-polyethylene oxide hydrogel was formed with one hour.

### Experimental Example 1

The chitosan methacrylate-polyethylene oxide hydrogel and the chitosan acrylate-polyethylene oxide hydrogel according to Examples 1 and 3 and a chitosan sample were evaluated by NMR. After the evaluation, it could be seen that acrylate and methacrylate were chemically bound to chitosan (see FIG. 7).

### Experimental Example 2

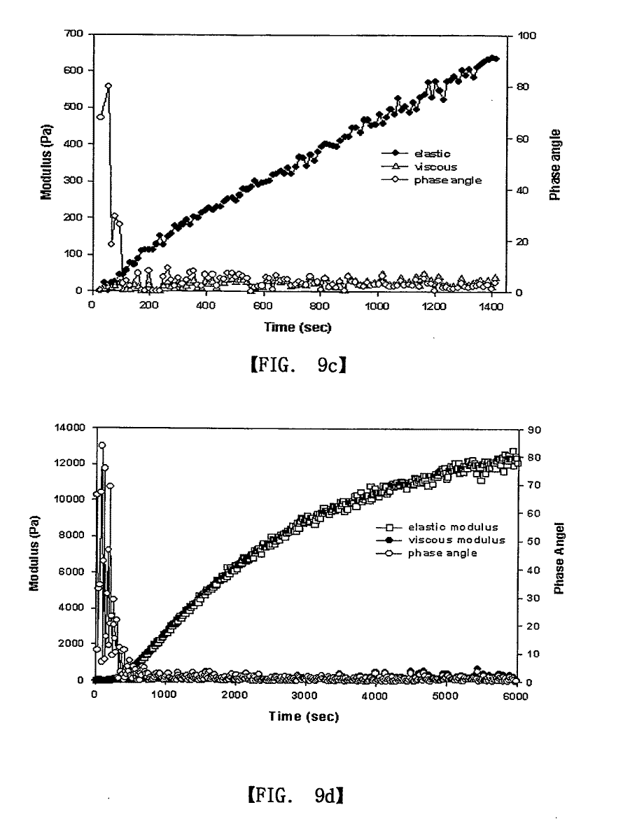

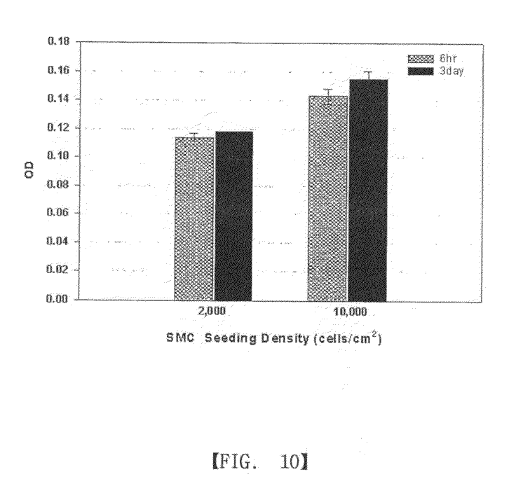

The chitosan-methacrylate solution according to Experimental Example 1 was mixed with a polyethylene oxide solution, and the resultant mixture was evaluated by using a rheometer. After the evaluation, it could be seen that a chitosan methacrylate-polyethylene oxide hydrogel started to be formed within 1 minute by observing variations in viscosity and elasticity (see FIG. 9-a).

### Experimental Example 3

A solution formed by mixing a mixed solution containing 75% of chitosan-acrylate and 25% of hyaluronic acid-aminopropyl methacrylate with a polyethylene oxide solution according to Example 8 was evaluated by using a rheometer with the lapse of time. After the evaluation, it could be seen that a chitosan acrylate-hyaluronic acid methacrylate-polyethylene oxide hydrogel started to be formed within 1 minute (see FIG. 9-b).

### Experimental Example 4

A solution formed by mixing a mixed solution containing 50% of chitosan-acrylate and 50% of hyaluronic acid-aminopropyl methacrylate with a polyethylene oxide solution according to Example 9 was evaluated by using a rheometer with the lapse of time. After the evaluation, it could be seen that a chitosan acrylate-polyethylene oxide methacrylate hydrogel started to be formed within 5 minutes (see FIG. 9-c).

### Experimental Example 5

A solution formed by mixing 100% of hyaluronic acid acrylate with a polyethylene oxide solution in Example 21 was evaluated by using a rheometer with the lapse of time. After the evaluation, it could be seen that a hyaluronic acid acrylate-polyethylene oxide hydrogel started to be formed within 1 minute (see FIG. 9-d).

### Experimental Example 6

Smooth muscle cells were cultured in vitro on the surface of the chitosan methacrylate-polyethylene oxide hydrogel obtained from Step 2 of Example 1 at a concentration of 2,000 and 10,000 cells/cm² for 6 hours and 3 days, and cell adhesion characteristics were observed by using an optical microscope. Then, cell counting kit-8 was added thereto for fluorometric detection and cell proliferation characteristics were evaluated by using a microplate reader. After the evaluation, it was observed that optical density (OD) increased. This indicates that living cells proliferate on the surface of the hydrogel.

### Experimental Example 7

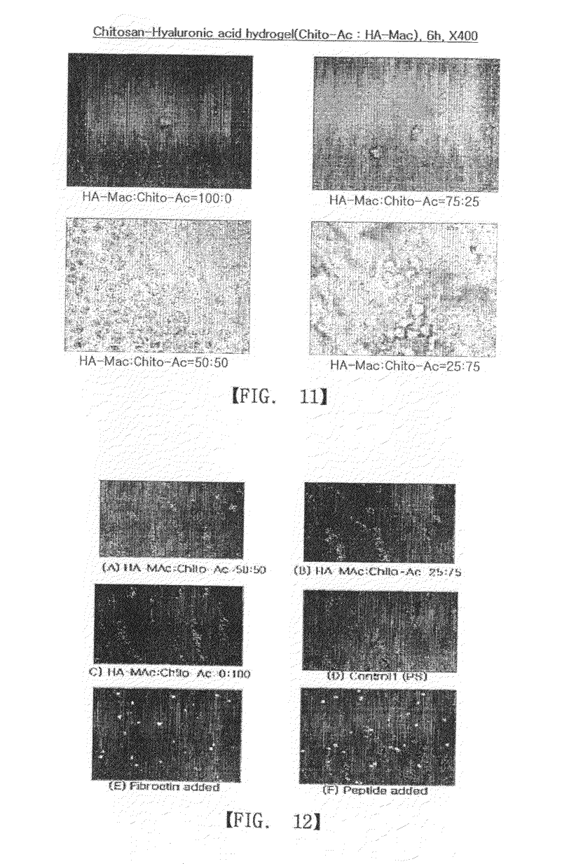

The 100% hyaluronic acid-methacrylate hydrogel, the mixed hydrogel of 75% hyaluronic acid-methacrylate/25% chitosan acrylate, the mixed hydrogel of 50% hyaluronic acid-methacrylate/50% chitosan-acrylate, and the mixed hydrogel of 25% hyaluronic acid-methacrylate/75% of chitosan-acrylate according to Examples 4 and 8-10 were used to culture smooth muscle cells in a cell culture system under the conditions of 37° C., 5% CO₂ for 6 hours. Each hydrogel was observed for cell proliferation and adhesion characteristics. After the observation, it could be seen that each hydrogel showed different cell adhesion characteristics (see FIG. 11).

### Experimental Example 8

The mixed hydrogel of 50% hyaluronic acid-methacrylate/50% chitosan-acrylate according to Example 9, the mixed hydrogel of 25% hyaluronic acid-methacrylate/75% chitosan-acrylate according to Example 10, and the 100% chitosan-acrylate hydrogel according to Example 3 were used to culture smooth muscle cells in a cell culture system having polystyrene cell culture flasks under the conditions of 37° C., 5% CO₂ for 6 hours. Each hydrogel was observed for cell proliferation and adhesion characteristics. After the observation, it could be seen that each hydrogel showed different cell adhesion characteristics (see FIG. 11).

### Experimental Example 9

The mixed solution containing hyaluronic acid methacrylate (50%) and chitosan acrylate (50%) according to Example 9 was further mixed with a polyethylene oxide solution containing 0.2% (w/w) of fibronectin to form a hyaluronic acid methacrylate-chitosan acrylate-polyethylene oxide hydrogel containing fibronectin. In vitro cell culture was carried out in a cell culture system under the conditions of 37° C., 5% CO₂ for a period of time up to one week. Then, cell proliferation and adhesion characteristics were observed. After the observation, it could be seen that the fibronectin-containing hydrogel showed improved cell adhesion characteristics (FIG. 12-E).

### Experimental Example 10

The mixed solution containing hyaluronic acid methacrylate (50%) and chitosan acrylate (50%) according to Example 9 was further mixed with a polyethylene oxide solution containing 0.2% (w/w) of CGRGDGC peptide to form a hyaluronic acid methacrylate-chitosan acrylate-polyethylene oxide hydrogel containing CGRGDGC peptide. In vitro cell culture was carried out in a cell culture system under the conditions of 37° C., 5% CO₂ for a period of time up to one week. Then, cell proliferation and adhesion characteristics were observed. After the observation, it could be seen that the CGRGDGC peptide-containing hydrogel showed improved cell adhesion characteristics (FIG. 12-F).

### Experimental Example 11

Cells were incorporated into the chitosan methacrylate-chitosan acrylate-polyethylene oxide-collagen hydrogel in the same manner as described in Example 16. In vitro cell culture was carried out in a cell culture system under the conditions of 37° C., 5% CO₂ for a period of time up to one week. Then, cell proliferation and adhesion characteristics were observed. After the observation, it could be seen that the hydrogel showed improved compatibility to the cells.

### Experimental Example 12

Cell adhesion and proliferation characteristics were evaluated in the same manner as described in Experimental Example 6, except that a hyaluronic acid methacrylate-chitosan acrylate-polyethylene oxide hydrogel was used instead of the chitosan methacrylate-polyethylene oxide hydrogel.

### Experimental Example 13

The hyaluronic acid-adipic acid hydrazide tert-butyl hydrazide and the hyaluronic acid-adipic acid acrylate compounds obtained from Example 21 were analyzed by NMR. The products were identified through the specific peaks of each compound by using hyaluronic acid as a reference compound (see FIG. 8-a, 8-b and 8-c).

### Experimental Example 14

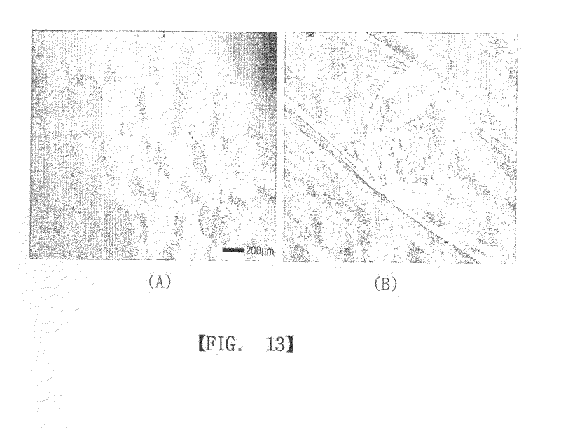

A mixture, formed by mixing the hyaluronic acid-acrylate (50%) according to Example 21 with the hyaluronic acid-methacrylate (50%) according to Example 4, was further mixed with a polyethylene oxide solution in a ratio of 1:1 to provide a mixed solution. Next, 1 mL of the mixed solution was introduced into a 20 mL syringe and was gradually added dropwise to 80 mL of dichloromethane solvent by using a syringe pump. At the same time, the hyaluronic acid-polyethylene oxide solution was stirred by suing a magnetic stirrer under 3,500 rpm. Then, a surfactant was gradually added thereto to perform a reaction, and the resultant product was filtered off by using a funnel, followed by freeze-drying. The dried sample was hydrated in an aqueous solution, and then observed by using an optical microscope (FIG. 13-A) and an electron microscope (FIG. 13-B). After the observation, it could be seen that microbeads having a size of 150˜200 μm were formed.

### INDUSTRIAL APPLICABILITY

As can be seen from the foregoing, the present invention provides a drug/cell-containing hydrogel with a different chitosan/hyaluronic acid ratio. The hydrogel can be used for regenerating an artificial organ for tissue engineering, producing a dressing material for treating a burn or a cosmetic dressing material, or for providing a drug delivery carrier. In such applications, the hydrogel can accomplish efficient drug delivery and stimulate tissue regeneration according to the biodegradation of the hydrogel. For example, when a mixed solution containing chitosan-acrylate and hyaluronic acid-methacrylate is sprayed in combination with a thiol group-containing polyethylene oxide solution onto the site of a burn or wound, a hydrogel is formed instantaneously or in a controlled time so that the site of a burn or wound can be treated. In a variant, cells are incorporated into a polyethylene oxide solution, and the solution is mixed with chitosan-acrylate, chitosan-methacrylate or a mixture thereof, or hyaluronic acid-acrylate, hyaluronic acid-methacrylate or a mixture thereof. Then, the resultant solution is sprayed by using a syringe. The hydrogel according to the present invention can maximize the unique characteristics of chitosan and hyaluronic acid. At the same time, it is possible to obtain a hydrogel having diverse physical properties in a desired time by controlling the ratio of methacrylate/acrylate. The hydrogel can be applied to a scaffold for tissue engineering, which can recover the tissue of a wound site having a complicated shape. Additionally, a bioactive substance can be incorporated into the hydrogel instead of the cells, so that the hydrogel can be used as a carrier for a drug capable of tissue regeneration or wound healing. Since a hydrogel can be formed in a predetermined time simply by mixing two kinds of solutions, the hydrogel can be provided in the form of two separate spray containers each containing one of the solutions. When spraying the solutions at the same time, the solutions are mixed to form a hydrogel. In this manner, treatment of the site of a wound can be accomplished. Since the hydrogel has excellent biocompatibility, it can be also used as filler for plastic surgery. In another variant, the hydrogel can be used as a cell/tissue adhesion barrier for preventing cell adhesion to tissues after a surgical operation by increasing the proportion of polyethylene oxide when preparing a chitosan-hyaluronic acid-polyethylene oxide hydrogel.

Although several preferred embodiments of the present invention have been described for illustrative purposes, those skilled in the art will appreciate that various modifications, additions and substitutions are possible, without departing from the scope and spirit of the invention as disclosed in the accompanying claims.

1.-30. (canceled)

31. A hydrogel characterized in that which is one of chitosan-polyethylene oxide-based, hyaluronic acid-polyethylene oxide-based, or chitosan-hyaluronic acid-polyethylene oxide-based hydrogels formed via covalent bonding between a mixture containing one of a chitosan acrylate derivative or a hyaluronic acid acrylate derivative crosslinked with an acrylate functional group-containing substance as well as one of a chitosan methacrylate derivative or a hyaluronic acid methacrylate derivative crosslinked with a methacrylate functional group-containing substance and a thiol functional group-containing substance.

32. The hydrogel in claim 31, wherein the hydrogel is provided as microbeads.

33. The hydrogel in claim 31, wherein the acrylate- or methacrylate functional group-containing substance is selected from the group consisting of:
acrylic acid, methacrylic acid, adipic acid hydrazide diamide acrylate, acrylamide, methacrylamide, alkyl-(meth)acrylamide, N-mono-(meth)acrylamide, N,N-di-C₁-C₄ alkyl-(meth)acrylamide, N-butyl(meth)acrylate, methyl(meth)acrylate, ethyl(meth)acrylate, isobornyl(meth)acrylate, cyclohexyl(meth)acrylate, hydroxyethylacrylate, hydroxyethyl methacrylate, hydroxypropyl acrylate, hydroxypropyl methacrylate, hydroxybutyl acrylate, N-(2-hydroxyethyl)acrylamide, N-methyl acrylamide, N-butoxymethyl acrylamide, N-methoxymethylacrylamide, N-methoxy methylmethacrylamide, 2-acrylamidoglycolic acid, and 2-carboxyethyl acrylate.

34. The hydrogel in claim 33, wherein the acrylate or methacrylate functional group-containing substance is selected from the group consisting of acrylamide, methacrylamide, allyl amine, adipic acid hydrazide hydrazide amide acrylate, and aminopropyl methacrylate.

35. The hydrogel in claim 34, wherein the thiol functional group-containing substance is a cysteine-containing peptide or protein in addition to polyethylene oxide.

36. The hydrogel in claim 33, which is for use in inducing tissue regeneration.

37. A bioactive substance delivery carrier comprising:
a bioactive substance supported on a hydrogel for use in inducing tissue regeneration, wherein the hydrogel characterized in that which is one of chitosan-polyethylene oxide-based, hyaluronic acid-polyethylene oxide-based, or chitosan-hyaluronic acid-polyethylene oxide-based hydrogels formed via covalent bonding between a mixture containing one of a chitosan acrylate derivative or a hyaluronic acid acrylate derivative crosslinked with an acrylate functional group-containing substance as well as one of a chitosan methacrylate derivative or a hyaluronic acid methacrylate derivative crosslinked with a methacrylate functional group-containing substance and a thiol functional group-containing substance.

38. The bioactive substance delivery carrier in claim 37, wherein the bioactive substance is selected from the group consisting of organic compounds, extracts, proteins, peptides, nucleic acids, extracellular matrix materials, cells, and inorganic compounds.

39. The bioactive substance delivery carrier in claim 38, wherein the organic compound is selected from the group consisting of antibiotics, anti-cancer agents, anti-inflammatory agents, anti-viral agents, antibacterial agents and hormones.

40. The bioactive substance delivery carrier in claim 38, wherein the protein is selected from the group consisting of hormones, cytokines, enzymes, antibodies, growth factors, transcription control factors, blood factors, vaccines, structural proteins, ligand proteins, receptors, cell surface antigens and receptor antagonists.

41. The bioactive substance delivery carrier in claim 38, wherein the extracellular matrix material is selected from the group consisting of collagen, fibronectin, gelatin, elastin, osteocalcin, fibrinogen, fibromodulin, tenascin, laminin, osteopontin, osteonectin, perlecan, versican, von Willebrand factor, fibrin and vitronectin.

42. The bioactive substance delivery carrier in claim 38, wherein the cell is selected from the group consisting of fibroblasts, vascular endothelial cells, smooth muscle cells, nerve cells, bone cells, dermal cells, chondrocytes, Schwann cells and stem cells.

43. The bioactive substance delivery carrier in claim 38, wherein the inorganic compound is selected from the group consisting of particles comprising hydroxyapatite, tricalcium phosphate and a mixture of hydroxyapatite-tricalcium phosphate, and the above inorganic compounds coated with proteins.

44. A method for preparing hyaluronic acid acrylate-polyethylene oxide hydrogel, the method comprising the steps of:
(a) protecting one end of dihydrazide;
(b) forming a hyaluronic acid-hydrazide tert-butyl hydrazide compound, one end of which is protected;
(c) removing the amine-protecting tert-butyl group from the hyaluronic acid-hydrazide tert-butyl hydrazide to provide hyaluronic acid hydrazide;
(d) forming hyaluronic acid-acrylate from the hyaluronic acid-hydrazide; and
(e) reacting the hyaluronic acid-acrylate with polyethylene oxide to provide a hyaluronic acid acrylate-polyethylene oxide hydrogel.

45. The method in claim 44, wherein the step of protecting one end of dihydrazide is carried out by chemically combining di-tert-butyl dicarbonate with a compound selected from the group consisting of adipic acid dihydrazide, oxalic acid dihydrazide, oxalyl dihydrazide, succinic acid dihydrazide, glutaric acid dihydrazide and ethylmalonic acid dihydrazide.

46. A hyaluronic acid (meth)acrylate compound, which is obtained by reacting the amine-protected compound as defined in claim 44 with hyaluronic acid, and by combining hyaluronic acid hydrazide, from which tert-butyl group is removed, with a (meth)acrylate compound. with a (meth)acrylate compound.
